(19) United States
(12) Patent Application Publication
(10) Pub. No.: US 2017/0169160 A1
(43) Pub. Date: Jun. 15, 2017

(54) VARIANT ANNOTATION, ANALYSIS AND SELECTION TOOL

(71) Applicants: Board of Regents, The University of Texas System, Austin, TX (US); University of Utah, Salt Lake City, UT (US)

(72) Inventors: Hao Hu, Houston, TX (US); Chad Huff, Houston, TX (US)

(21) Appl. No.: 15/342,927

(22) Filed: Nov. 3, 2016

Related U.S. Application Data
(63) Continuation of application No. PCT/US2015/029318, filed on May 5, 2015.
(60) Provisional application No. 61/988,826, filed on May 5, 2014.

(51) Int. Cl. G06F 19/18; G06F 19/24; C12Q 1/68; G06F 19/22

(52) U.S. Cl. CPC G06F 19/18; G06F 19/22; G06F 19/24; C12Q 1/6876; C12Q 2600/156

(57) ABSTRACT

Disclosed herein are methods for detecting and/or prioritizing phenotype-causing genomic variants and related software tools. A method of the present disclosure comprises (a) computer processing instructions that prioritize generic variants combining (i) variant frequency, (ii) one or more sequence characteristics and (iii) a summing procedure and (b) automatically identifying and reporting the phenotype-causing genetic variants. The method incorporates pedigree data summarized by a log odds (LOD) score in each family.

### CROSS-REFERENCE

This application is a continuation application of International Patent Application No. PCT/US2015/029318, filed May 5, 2015, which application claims the benefit of U.S. Provisional Patent Application Ser. No. 61/988,826, filed May 5, 2014, which is incorporated by reference herein in its entirety.

### STATEMENT AS TO FEDERALLY SPONSORED RESEARCH

This invention was made with government support under Grant RC2HG005619 awarded by the NIH National Human Genome Research Institute (NHGRI). The government has certain rights in the invention.

### BACKGROUND

Manual analysis of personal genome sequences is a massive, labor-intensive task. Although much progress is being made in deoxyribonucleic acid (DNA) sequence read alignment and variant calling, little software yet exists for the automated analysis of personal genome sequences. Indeed, the ability to automatically annotate variants, to combine data from multiple projects, and to recover subsets of annotated variants for diverse downstream analyses is becoming a critical analysis bottleneck.

Researchers are now faced with multiple whole genome sequences, each of which has been estimated to contain around 4 million variants. This creates a need to efficiently prioritize variants so as to efficiently allocate resources for further downstream analysis, such as external sequence validation, additional biochemical validation experiments, further target validation such as that performed routinely in a typical Biotech/Pharma discovery effort, or in general additional variant validation. Such relevant variants are also called phenotype-causing genetic variants.

### SUMMARY

In light of at least some of the limitations of current methods and systems, recognized herein is the need for improved methods and systems for genomic analysis.

The present disclosure provides methods and systems that can automatically annotate variants, combine data from multiple projects, and recover subsets of annotated variants for diverse downstream analyses. Methods and systems provided herein can efficiently prioritize variants so as to efficiently and effectively allocate resources for further downstream analysis, such as external sequence validation, additional biochemical validation experiments, further target validation, and additional variant validation.

An aspect of the present disclosure provides a method for identifying phenotype-causing genetic variants comprising (a) computer processing instructions that prioritize genetic variants by combining (i) variant frequency, (ii) one or more sequence characteristics and (iii) a summing procedure; (b) automatically identifying and reporting the phenotype-causing genetic variants, wherein the method incorporates pedigree data summarized by a log odds (LOD) score in each family.

One embodiment provides a method comprising excluding both de novo mutations and sequencing errors, for genomes with an error rate of approximately 1 in 100,000, approximately 99.9% of all Mendelian inheritance errors are genotyping errors. Another embodiment provides a method wherein the genetic variants used to calculate LOD scores are determined a priori using the method disclosed herein. Another embodiment provides a method wherein a Mendelian inheritance error rate is included in the LOD score calculation.

Another aspect of the present disclosure provides a computer system for identifying phenotype-causing genome sequence variants, comprising: (a) computer memory storing a plurality of genome sequence variants from an assay performed on a biological sample of a subject exhibiting a phenotype, which subject is from a pedigree that comprises the subject and one or more individuals related thereto that do not exhibit the phenotype; and (b) a computer processor coupled to the computer memory, wherein the computer processor is programmed to: (i) group the genome sequence variants from the computer memory into user-defined features; (ii) evaluate a potential severity of the genome sequence variants by estimating variant frequency and/or amino acid substitution frequency; (iii) calculate by a summing procedure a likelihood ratio of the genome sequence variants occurring within the user-defined features for the subject as compared to the genome sequence variants occurring within the user defined features in biological samples of control subject(s) not exhibiting the phenotype; (iv) determine log odds (LOD) scores for the genome sequence variants, wherein a given LOD score is indicative of a given genome sequence variant being causative or associated with the phenotype; (v) prioritize the genome sequence variants by at least the LOD scores, thereby providing prioritized genome sequence variants; and (vi) report the prioritized genome sequence variants.

In some embodiments of aspects provided herein, the computer processor is programmed to evaluate a potential severity of the genome sequence variants by estimating variant frequency and amino acid substitution frequency. In some embodiments of aspects provided herein, the user-defined features comprise one or more of a gene, an exon, an intron, a protein coding sequence, a splice site, a promoter, a regulatory sequence, a protein binding site, an enhancer, and a repressor. In some embodiments of aspects provided herein, the genome sequence variants comprise coding and non-coding genome sequence variants, and the computer processor is programmed to (i) score both of the coding and non-coding genome sequence variants; and (ii) evaluate a cumulative impact of both types of the genome sequence variants simultaneously. In some embodiments of aspects provided herein, the programmed computer processor is programmed to incorporate both rare and common genomic sequence variants to identify variants that are associated with common phenotypes. In some embodiments of aspects provided herein, the phenotype is a disease. In some embodiments of aspects provided herein, the computer system further comprises a communication interface for obtaining genetic information containing the genome sequence variants of the subject. In some embodiments of aspects provided herein, the computer processor is programmed to use the genome sequence variants to analyze the genetic information of the subject to identify another phenotype in the subject. In some embodiments of aspects provided herein, the computer processor is programmed to generate a report that is indicative of the another phenotype in the subject. In some embodiments of aspects provided herein, the computer processor is programmed to use the prioritized genome sequence variants to identify a disease associated with the phenotype or the another phenotype in the subject. In some embodiments of aspects provided herein, the disease is a genetic disorder. In some embodiments of aspects provided herein, the computer processor is programmed to recommend a therapeutic intervention for the disease. In some embodiments of aspects provided herein, the report is provided for display on a user interface on an electronic display. In some embodiments of aspects provided herein, the computer processor is programmed to format the report for display on the user interface.

Another aspect of the present disclosure provides a method for identifying phenotype-causing genome sequence variants, comprising: (a) using a programmed computer processor to (i) group genome sequence variants within user-defined features, which genome sequence variants are from an assay performed on a biological sample of a subject exhibiting a phenotype, which subject is from a pedigree that comprises the subject and one or more individuals related thereto that do not exhibit the phenotype, and (ii) evaluate a potential severity of the genome sequence variants by estimating variant frequency and/or amino acid substitution frequency; (b) calculating by a summing procedure a likelihood ratio of the genome sequence variants occurring within the user-defined features for the subject as compared to the genome sequence variants occurring within the user defined features in biological samples of control subject(s) not exhibiting the phenotype; (c) determining log odds (LOD) scores for the genome sequence variants, wherein a given LOD score is indicative of a given genome sequence variant being causative or associated with the phenotype; (d) prioritizing the genome sequence variants by at least the LOD scores; and (e) reporting the genome sequence variants prioritized in (d).

In some embodiments of aspects provided herein, the computer processor evaluates a potential severity of the genome sequence variants by estimating variant frequency and amino acid substitution frequency. In some embodiments of aspects provided herein, the genome sequence variants are both rare and common genome sequence variants. In some embodiments of aspects provided herein the phenotype is a common disease. In some embodiments of aspects provided herein, the genome sequence variants are rare genome sequence variants. In some embodiments of aspects provided herein, the phenotype is a rare disease. In some embodiments of aspects provided herein, the method further comprises using genome sequence variants to identify another phenotype in the subject. In some embodiments of aspects provided herein, the method further comprises generating a report that is indicative of the another phenotype in the subject. In some embodiments of aspects provided herein, the method further comprises using the prioritized genome sequence variants to identify a disease associated with the phenotype in the subject. In some embodiments of aspects provided herein, the disease is a genetic disorder. In some embodiments of aspects provided herein, the method further comprises recommending a therapeutic intervention for the disease. In some embodiments of aspects provided herein, the method further comprises incorporating a genetic profile of a single individual, wherein the genetic profile comprises single-nucleotide polymorphisms, a set of one or more genes, an exome or a genome, a genomic profile of one or more individuals analyzed together, or genomic profiles from individuals from a family. In some embodiments of aspects provided herein, the prioritizing the genome sequence variants by at least the LOD scores has a statistical power at least 10 times greater than prioritizing the genome sequence variants without the LOD scores.

In some embodiments of aspects provided herein, the summing procedure assesses genome sequence variants in both coding and non-coding regions of a genome of the subject within a given user-defined feature of the user-defined features to determine the likelihood ratio. In some embodiments of aspects provided herein, the user-defined features comprise low-complexity and repetitive genome sequences. In some embodiments of aspects provided herein, an individual in the pedigree contributes to both the LOD scores and the calculating. In some embodiments of aspects provided herein, the determining of the LOD scores for the genome sequence variants utilizes phasing information of the genome sequence variants. In some embodiments of aspects provided herein, the subject exhibiting the phenotype and the individuals not exhibiting the phenotype are included in a target and a background database, respectively. In some embodiments of aspects provided herein, the target and background databases comprise genome sequence variants of the subject exhibiting the phenotype and the individuals not exhibiting the phenotype. In some embodiments of aspects provided herein, the target and background databases comprise information on family members of the subject, wherein the information comprises whether the family members have exhibited the phenotype. In some embodiments of aspects provided herein, (i) the genome sequence variants are prioritized using a trained algorithm or (ii) the prioritized genome sequence variants are used to generate the trained algorithm. In some embodiments of aspects provided herein, the method further comprises comparing the genome sequence variant data in a background database and a target database. In some embodiments of aspects provided herein, the background and target databases comprise the genome sequence variants of the individuals not exhibiting the phenotype and the subject exhibiting the phenotype, respectively.

In some embodiments of aspects provided herein, determining the LOD scores further comprises calculating a likelihood of a null model and an alternative model, wherein the models assume independence between nucleotide sites. In some embodiments of aspects provided herein, a significance of the likelihood is determined by permuting to control for linkage disequilibrium. In some embodiments of aspects provided herein, the method further comprises performing a nested composite likelihood ratio (CLR) test that depends only on differences in allele frequencies between individuals exhibiting the phenotype and individuals not exhibiting the phenotype to further quantify an extent to which each of the genome sequence variants is causative or associated with the phenotype. In some embodiments of aspects provided herein, the genome sequence variants comprise rare minor alleles within one or more of the user-defined features, and (i) a total number of the rare minor alleles within the user-defined features among the individuals not exhibiting the phenotype is determined; (ii) a total number of the rare minor alleles within the user-defined features for the subject exhibiting the phenotype is determined; and (iii) calculating a likelihood that the user-defined features are associated with the phenotype based on the total number of the rare minor alleles from (i) and (ii). In some embodiments of aspects provided herein, the rare minor alleles are observed fewer than five times in a target database. In some embodiments of aspects provided herein, the method further comprises determining k as a number of uncollapsed variant sites among nᵢ^{U} unaffected and nᵢ^{A} affected individuals, with nᵢ equal to nᵢ^{U}+nᵢ^{A}, wherein lₖ₊₁ . . . lₖ₊ₘ, equal a number of collapsed variant sites within m collapsing categories labeled k+1 to m, and let l₁ . . . lₖ equal 1 to identify groups of variants, within each group an effect on the phenotype is expected to be similar based on a priori evidence. In some embodiments of aspects provided herein, the method comprises determining Xᵢ, Xᵢ^{U}, and Xᵢ^{A} as a number of copies of the minor allele(s) at variant site i or collapsing category i among all individuals, unaffected individuals, and affected individuals, respectively, for which an effect on the phenotype is independently estimated.

In some embodiments of aspects provided herein, the calculating by the summing procedure the likelihood ratio further comprises performing the calculating in accordance with:

$$λ = ln ( \frac{{L}_{Null}}{{L}_{Alt}} ) = {\sum }_{i = 1}^{k + m} ln [ \frac{{( {\hat{p}}_{1} )}^{{X}_{i}} {( 1 - {\hat{p}}_{i} )}^{2 {l}_{i} {n}_{i} - {X}_{i}}}{{( {\hat{p}}_{i}^{U} )}^{{X}_{i}^{U}} {( 1 - {\hat{p}}_{i}^{U} )}^{2 {l}_{i} {n}_{i}^{U} - {X}_{i}^{U}} {( {\hat{p}}_{i}^{A} )}^{{X}_{i}^{A}} {( 1 - {\hat{p}}_{i}^{A} )}^{2 {l}_{i} {n}_{i}^{A} - {X}_{i}^{A}}} ] ,$$

wherein pᵢ, pᵢ^{U}, and pᵢ^{A} equal maximum likelihood estimates for a frequency of minor allele(s) within the genome sequence variants at site i in a genome for (i) individuals exhibiting the phenotype and the individuals not exhibiting the phenotype; (ii) the individuals not exhibiting the phenotype; and (iii) the individuals exhibiting the phenotype, respectively, and wherein the site i is a specific nucleotide base, a user-defined feature from the user-defined features, or collapsed category i of the user-defined features among all individuals, unaffected individuals, and affected individuals, respectively. In some embodiments of aspects provided herein, the maximum likelihood estimates are equal to an observed frequency of the minor allele(s).

In some embodiments of aspects provided herein, the method further comprises reporting logₑ of a χ² p-value as a score to provide a statistic for rapid prioritization of disease-gene candidates wherein variant sites are unlinked to provide a measure of an overall evidence of phenotype association within each of the user-defined features, wherein −2λ. approximately follows a χ² distribution with k+m degrees of freedom. In some embodiments of aspects provided herein, the minor allele(s) comprise one or more of indels, splice-site variants, synonymous variants, and non-coding variants. In some embodiments of aspects provided herein, the method further comprises incorporating into the statistic for rapid prioritization information about a severity of amino acid changes to assess evidence of to assess evidence of an association of the genome sequence variants to the phenotype.

In some embodiments of aspects provided herein, the method further comprises including an additional parameter in a null and alternative model to estimate a phenotype effect size for each variant site or collapsed category of the user-defined features. In some embodiments of aspects provided herein a log of the likelihood ratio A is calculated as

$$λ = ln ( \frac{{L}_{Null}}{{L}_{Alt}} ) = {\sum }_{i = 1}^{k + m} ln [ \frac{{{h}_{i} ( {\hat{p}}_{i} )}^{{X}_{i}} {( 1 - {\hat{p}}_{i} )}^{2 {l}_{i} {n}_{i} - {X}_{i}}}{{{a}_{i} ( {\hat{p}}_{i}^{U} )}^{{X}_{i}^{U}} {( 1 - {\hat{p}}_{i}^{U} )}^{2 {l}_{i} {n}_{i}^{U} - {X}_{i}^{U}} {( {\hat{p}}_{i}^{A} )}^{{X}_{i}^{A}} {( 1 - {\hat{p}}_{i}^{A} )}^{2 {l}_{i} {n}_{i}^{A} - {X}_{i}^{A}}} ] . ,$$

wherein the parameter hᵢ in a null model, L_{Null}, is a likelihood that an amino acid change caused by the genome sequence variant does not contribute to a disease risk, wherein the parameter aᵢ in an alternative model, L_{Alt}, is a likelihood that an amino acid change caused by the genome sequence variant contributes to a disease risk. In some embodiments of aspects provided herein, the genome sequence variants within the coding regions can be synonymous or non-synonymous.

In some embodiments of aspects provided herein, the method further comprises estimating hᵢ by setting it equal to a proportion of a corresponding type of amino acid change in a population background. In some embodiments of aspects provided herein, the method further comprises estimating aᵢ by setting it equal to a proportion of a corresponding type of amino acid change among all disease-causing mutations in a selected study population.

In some embodiments of aspects provided herein, the summing procedure assesses the genome sequence variants in the non-coding regions and synonymous variants in the coding regions by estimating a relative impact of non-coding and synonymous variants using vertebrate-to-human genome multiple alignments. In some embodiments of aspects provided herein, estimating relative impact of the synonymous variants further comprises determining, for each codon in a human genome, a frequency with which the codon is replaced with a different codon in genome(s) of one or more other primate species.

In some embodiments of aspects provided herein, the LOD scores are determined assuming a genome sequence variant that causes the subject to exhibit the phenotype is inherited under a recessive inheritance model, a recessive with complete penetrance inheritance model, a monogenic recessive inheritance model, or a combination thereof. In some embodiments of aspects provided herein, the method further comprises excluding the genome sequence variants within one or more of the user-defined features from the likelihood calculation. In some embodiments of aspects provided herein, the genome sequence variants that are not present in two or more members of the pedigree of the subject are excluded. In some embodiments of aspects provided herein,

$$λ = ln ( \frac{{L}_{Null}}{{L}_{Alt}} ) = {\sum }_{i = 1}^{k + m} ln [ \frac{{{h}_{i} ( {\hat{p}}_{i} )}^{{X}_{i}} {( 1 - {\hat{p}}_{i} )}^{2 {l}_{i} {n}_{i} - {X}_{i}}}{{{a}_{i} ( {\hat{p}}_{i}^{U} )}^{{X}_{i}^{U}} {( 1 - {\hat{p}}_{i}^{U} )}^{2 {l}_{i} {n}_{i}^{U} - {X}_{i}^{U}} {( {\hat{p}}_{i}^{A} )}^{{X}_{i}^{A}} {( 1 - {\hat{p}}_{i}^{A} )}^{2 {l}_{i} {n}_{i}^{A} - {X}_{i}^{A}}} ] .$$

In some embodiments of aspects provided herein, the method further comprises determining which genome sequence variants are used to determine the LOD scores based on genome sequence variants with maximum likelihood ratios determined in (a) and (b). In some embodiments of aspects provided herein, the method further comprises constraining an estimated recombination rate to 0 in both null and alternative models for determining the LOD scores. In some embodiments of aspects provided herein, a second latent locus is included in the determining the LOD scores using

Pₙᵤₗₗ(g_{c},gₗ,p|ρ_{c},ρₗ,f_{c},fₗ)=P(g_{c}|f_{c})P(gₗ,p|ρₗ,fₗ).

In some embodiments of aspects provided herein, determining the LOD scores further comprises utilizing a rate of de novo mutation per meiosis in human genomes.

In some embodiments of aspects provided herein, the method further comprises incorporating a disease model that allows for recessive and compound heterozygote patterns of inheritance by estimating a Boolean risk vector of disease causality at each genome sequence variant, using a computational optimization technique. In some embodiments of aspects provided herein, the computational optimization technique comprises Markov Chain Monte Carlo. In some embodiments of aspects provided herein, the method further comprises optimizing:

L=ρᵣⁿ^{ₐ}(1−ρᵣ)ⁿ^{_{b}}ρₙⁿ^{_{c}}(1−ρₙ)ⁿ^{_{d}},

wherein “L” is a joint likelihood that the user-defined features contain two or more genome sequence variants that are associated with the phenotype, “ρᵣ” is a probability that an individual with a genotype associated with the phenotype exhibits the phenotype, “ρₐ” is a probability that an individual with a genotype not associated with the phenotype exhibits the phenotype; “nₐ” and “n_{b}” are total numbers of individuals exhibiting the phenotype and individuals not exhibiting the phenotype, each of which have a genotype associated with the phenotype, respectively; “n_{c}” and “n_{d}” are total numbers of individuals with a genotype not associated with the phenotype that exhibit the phenotype and individuals with a genotype not associated with the phenotype that do not exhibit the phenotype, respectively.

In some embodiments of aspects provided herein, the LOD scores for each of the genome sequence variants are first determined across each of two or more pedigrees before determining which of the genome sequence variants are used to determine the LOD scores in each of the pedigrees.

In some embodiments of aspects provided herein, the method further comprises determining a statistical significance of the likelihood ratio and the LOD scores using a combined permutation test and a gene drop simulation. In some embodiments of aspects provided herein, the permutation test estimates the statistical significance in the pedigree, wherein a founder in the pedigree may or may not have genome sequence variant data, by repeated sampling of a combined database of genome sequence variants from target and background genomes and randomly assigning the genome sequence variants from target and background genomes to the founder. In some embodiments of aspects provided herein, the permutation test randomly assigns the genome sequence variants of target or background individuals to a pedigree founder in permutation, wherein the subject was used to calculate the LOD scores in the pedigree. In some embodiments of aspects provided herein, the gene drop simulation randomly determines the genome sequence variants in non-founder members of the pedigree using Mendelian rules of inheritance. In some embodiments of aspects provided herein, de novo mutations and Mendelian inheritance errors are randomly introduced in the gene drop simulation to provide a capability of calculating statistical significance for de novo mutations. In some embodiments of aspects provided herein, a rate of the de novo mutations and the Mendelian inheritance errors is determined empirically using genome sequence variants from the pedigree to provide the capability of calculating statistical significance. In some embodiments of aspects provided herein, identity-by-descent information from genome sequence data of individuals within the pedigree is evaluated during the identifying phenotype-causing genome sequence variants.

Another aspect of the present disclosure provides a computer readable medium comprising machine executable code that, upon execution by one or more computer processors, implements any of the methods above or elsewhere herein.

Another aspect of the present disclosure provides a computer system comprising one or more computer processors and a computer readable medium coupled thereto. The computer readable medium comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “FIG.” and “FIGs.” herein), of which:

FIG. 1 provides a schematic diagram for the calculation of composite likelihood ratio test (CLRTₚ) scores in pedigree Variant Annotation, Analysis and Search Tool (pVAAST);

FIGS. 2A-2C show the number of families required by different analytical methods to correctly identify the gene associated with a condition with varying population attributable risk for under a dominant model (2A), a recessive model (2B), and a dominant model with de novo mutations (2C);

FIGS. 3A-3D show a number of families that may be used to identify a phenotype-causing gene for varying selection coefficients and number of informative meioses separating samples;

FIGS. 4A-4B show the ability of pVAAST to identify a phenotype-causing gene for a de novo mutation causing for a child with enteropathy;

FIGS. 5A-5B show the ability of pVAAST to identify a phenotype-causing gene for a cardiac septal defect in a four-generation pedigree;

FIGS. 6A-6B show pVAAST's ability to identify the phenotype-causing gene for a recessively inherited genetic defect causing Miller's Syndrome in a two-generation pedigree;

FIG. 7 shows an exemplary computer system that can be used for the analysis of genetic variants and pedigree information to identify phenotype-causing genotypes; and

FIGS. 8A-8H show the genome-wide ranking and log-odds (LOD) score of GATA4 in challenging situations of pedigree studies, (A-H) LOD scores and genome-wide rankings corresponding to differing levels of unknown phenotypes (A,B), degrees of penetrance (C,D), proportion of affected individuals being G296S mutation carriers (E,F) and number of informative meioses (G,H).

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Before the present methods are disclosed and described, it is to be understood that this disclosure is not limited to specific embodiments. It is also to be understood that the terminology used herein is for the purpose of describing particular embodiments only and is not intended to be limiting. The following description and examples illustrate some exemplary embodiments of the disclosure in detail. Those of skill in the art will recognize that there are numerous variations and modifications of this disclosure that are encompassed by its scope. Accordingly, the description of a certain exemplary embodiment should not be deemed to limit the scope of the present disclosure.

The term “subject,” as used herein, generally refers to an animal, such as a mammalian species (e.g., human) or avian (e.g., bird) species, or other organism, such as a plant. A subject can be a vertebrate, a mammal, a mouse, a primate, a simian or a human. Mammals include, but are not limited to, murines, simians, humans, farm animals, sport animals, and pets. A subject can be a healthy individual, an individual that has or is suspected of having a disease or a pre-disposition to the disease, or an individual that is in need of therapy or suspected of needing therapy. A subject can be a patient.

An “individual” can be of any species of interest that comprises genetic information. The individual can be a eukaryote, a prokaryote, or a virus. The individual can be an animal or a plant. The individual can be a human or non-human animal.

The term “sequencing,” as used herein, generally refers to methods and technologies for determining the sequence of nucleotide bases in one or more polynucleotides. The polynucleotides can be, for example, deoxyribonucleic acid (DNA) or ribonucleic acid (RNA), including variants or derivatives thereof (e.g., single stranded DNA). Sequencing can be performed by various systems currently available, such as, with limitation, a sequencing system by Illumina, Pacific Biosciences, Oxford Nanopore, or Life Technologies (Ion Torrent). Such devices may provide a plurality of raw genetic data corresponding to the genetic information of a subject (e.g., human), as generated by the device from a sample provided by the subject. In some situations, systems and methods provided herein may be used with proteomic information.

The present disclosure relates to methods for the identification of phenotype-causing variants. The methods can comprise the comparison of polynucleotide sequences between a case, or target cohort, and a background, or control, cohort. Phenotype-causing variants can be scored within the context of one or more features. Variants can be coding or non-coding variants. The methods can employ a feature-based approach to prioritization of variants. The feature-based approach can be an aggregative approach whereby all the variants within a given feature are considered for their cumulative impact upon the feature (e.g., a gene or gene product). Therefore, the method also allows for the identification of features such as genes or gene products. Prioritization can employ variant frequency information, sequence characteristics such as amino acid substitution effect information, phase information, pedigree information, disease inheritance models, or a combination thereof.

“Nucleic acid” and “polynucleotide” can be used interchangeably herein, and refer to both RNA and DNA, including cDNA, genomic DNA, synthetic DNA, and DNA or RNA containing nucleic acid analogs. Polynucleotides can have any three-dimensional structure. A nucleic acid can be double-stranded or single-stranded (e.g., a sense strand or an antisense strand). Non-limiting examples of polynucleotides include chromosomes, chromosome fragments, genes, intergenic regions, gene fragments, exons, introns, messenger RNA (mRNA), transfer RNA, ribosomal RNA, siRNA, micro-RNA, ribozymes, cDNA, recombinant polynucleotides, branched polynucleotides, nucleic acid probes and nucleic acid primers. A polynucleotide may contain unconventional or modified nucleotides.

“Nucleotides” are molecules that when joined together form the structural basis of polynucleotides, e.g., ribonucleic acids (RNA) and deoxyribonucleic acids (DNA). A “nucleotide sequence” is the sequence of nucleotides in a given polynucleotide. A nucleotide sequence can also be the complete or partial sequence of an individual's genome and can therefore encompass the sequence of multiple, physically distinct polynucleotides (e.g., chromosomes).

The “genome” of an individual member of a species can comprise that individual's complete set of chromosomes, including both coding and non-coding regions. Particular locations within the genome of a species are referred to as “loci,” “sites” or “features”. “Alleles” are varying forms of the genomic DNA located at a given site. In the case of a site where there are two distinct alleles in a species, referred to as “A” and “B,” each individual member of the species can have one of four possible combinations: AA; AB; BA; and BB. The first allele of each pair is inherited from one parent, and the second from the other.

The “genotype” of an individual at a specific site in the individual's genome refers to the specific combination of alleles that the individual has inherited. A “genetic profile” for an individual includes information about the individual's genotype at a collection of sites in the individual's genome. As such, a genetic profile is comprised of a set of data points, where each data point is the genotype of the individual at a particular site.

Genotype combinations with identical alleles (e.g., AA and BB) at a given site are referred to as “homozygous;” genotype combinations with different alleles (e.g., AB and BA) at that site are referred to as “heterozygous.” It should be noted that in determining the allele in a genome using standard techniques AB and BA cannot be differentiated, meaning it may be impossible to determine from which parent a certain allele has been inherited, given solely the genomic information of the individual tested. Moreover, variant AB parents can pass either variant A or variant B to their children. While such parents may not have a predisposition to develop a disease, their children may. For example, two variant AB parents can have children who are variant AA, variant AB, or variant BB. One of the two homozygotic combinations in this set of three variant combinations may be associated with a disease. Having advance knowledge of this possibility can allow potential parents to make the best possible decisions about their children's health.

An individual's genotype can include haplotype information. A “haplotype” is a combination of alleles that are inherited or transmitted together. “Phased genotypes” or “phased datasets” provide sequence information along a given chromosome and can be used to provide haplotype information.

The “phenotype” of an individual refers to one or more characteristics. An individual's phenotype can be driven by constituent proteins in the individual's “proteome,” which is the collection of all proteins produced by the cells comprising the individual and coded for in the individual's genome. The proteome can also be defined as the collection of all proteins expressed in a given cell type within an individual. A disease or disease-state can be a phenotype and can therefore be associated with the collection of atoms, molecules, macromolecules, cells, tissues, organs, structures, fluids, metabolic, respiratory, pulmonary, neurological, reproductive or other physiological function, reflexes, behaviors and other physical characteristics observable in the individual through various approaches.

In some cases, a given phenotype can be associated with a specific genotype. For example, an individual with a certain pair of alleles for the gene that encodes for a particular lipoprotein associated with lipid transport may exhibit a phenotype characterized by a susceptibility to a hyperlipidemous disorder that leads to heart disease.

The “background” or “background database” can be a collection of nucleotide sequences (e.g., one or more genes or gene fragments, one or more chromosomes or chromosome fragments, one or more genomes or genome fragments, one or more transcriptome sequences, etc.) and their variants (variant files) used to derive reference variant frequencies in the background sequences. The background database can contain any number of nucleotide sequences and can vary based upon the number of available sequences. The background database can contain about 1-10000, 1-5000, 1-2500, 1-1000, 1-500, 1-100, 1-50, 1-10, 10-10000, 10-5000, 10-2500, 10-1000, 10-500, 10-100, 10-50, 50-10000, 50-5000, 50-2500, 50-1000, 50-500, 50-100, 100-10000, 100-5000, 100-2500, 100-1000, 100-500, 500-10000, 500-5000, 500-2500, 500-1000, 1000-10000, 1000-5000, 1000-2500, 2500-10000, 2500-5000, or 5000-10000 sequences, or any included sub-range; for example, about 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 600, 700, 800, 900, 1000, 1250, 1500, 1750, 2000, 2500, 3000, 3500, 4000, 4500, 5000, 6000, 7000, 8000, 9000, 10000, or more sequences, or any intervening integer.

The “target” or “case” can be a collection of nucleotide sequences (e.g., one or more genes or gene fragments, one or more genomes or genome fragments, one or more transcriptome sequences, etc.) and their variants under study. The target can contain information from individuals that exhibit the phenotype under study. The target can be a personal genome sequence or collection of personal genome sequences. The personal genome sequence can be from an individual diagnosed with, suspected of having, or at increased risk for a disease. The target can be a tumor genome sequence. The target can be genetic sequences from plants or other species that have desirable characteristics.

The term “cohort” can be used to describe a collection of target or background sequences, and their variants, used in a given comparison. A cohort can include about 1-10000, 1-5000, 1-2500, 1-1000, 1-500, 1-100, 1-50, 1-10, 10-10000, 10-5000, 10-2500, 10-1000, 10-500, 10-100, 10-50, 50-10000, 50-5000, 50-2500, 50-1000, 50-500, 50-100, 100-10000, 100-5000, 100-2500, 100-1000, 100-500, 500-10000, 500-5000, 500-2500, 500-1000, 1000-10000, 1000-5000, 1000-2500, 2500-10000, 2500-5000, or 5000-10000 sequences, or any included sub-range; for example, about 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 600, 700, 800, 900, 1000, 1250, 1500, 1750, 2000, 2500, 3000, 3500, 4000, 4500, 5000, 6000, 7000, 8000, 9000, 10000, or more sequences, or any intervening integer.

A “variant” can be any change in an individual nucleotide sequence compared to a reference sequence. The reference sequence can be a single sequence, a cohort of reference sequences, or a consensus sequence derived from a cohort of reference sequences. An individual variant can be a coding variant or a non-coding variant. A variant wherein a single nucleotide within the individual sequence is changed in comparison to the reference sequence can be referred to as a single nucleotide polymorphism (SNP) or a single nucleotide variant (SNV) and these terms are used interchangeably herein. SNPs that occur in the protein coding regions of genes that give rise to the expression of variant or defective proteins are potentially the cause of a genetic-based disease. Even SNPs that occur in non-coding regions can result in altered mRNA and/or protein expression. Examples are SNPs that defective splicing at exon/intron junctions. An exon is any nucleotide sequence encoded by a gene that remains present in the final mature mRNA product of that gene after introns have been removed by RNA splicing. Introns are noncoding segments of an RNA transcript, or the DNA encoding it, that are spliced out before the RNA is translated into a protein. In the process by which genomic DNA is transcribed into messenger RNA, introns are often spliced out of pre-messenger RNA transcripts to yield messenger RNA. A SNP can be in a coding region or a non-coding region. A SNP in a coding region can be a silent mutation, otherwise known as a synonymous mutation, wherein an encoded amino acid is not changed due to the variant. A SNP in a coding region can be a missense mutation, wherein an encoded amino acid is changed due to the variant. A SNP in a coding region can also be a nonsense mutation, wherein the variant introduces a premature stop codon. A variant can include an insertion or deletion (INDEL) of one or more nucleotides. An INDEL can be a frame-shift mutation, which can significantly alter a gene product. An INDEL can be a splice-site mutation. A variant can be a large-scale mutation in a chromosome structure; for example, a copy-number variant caused by an amplification or duplication of one or more genes or chromosome regions or a deletion of one or more genes or chromosomal regions; or a translocation causing the interchange of genetic parts from non-homologous chromosomes, an interstitial deletion, or an inversion.

Variants can be provided in a variant file, for example, a genome variant file (GVF) or a variant call format (VCF) file. According to the methods disclosed herein, tools can be provided to convert a variant file provided in one format to another more preferred format. A variant file can comprise frequency information on the included variants.

A “feature” can be any span or a collection of spans within a nucleotide sequence (e.g., a genome or transcriptome sequence). A feature can comprise a genome or genome fragment, one or more chromosomes or chromosome fragments, one or more genes or gene fragments, one or more transcripts or transcript fragments, one or more exons or exon fragments, one or more introns or intron fragments, one or more splice sites, one or more regulatory elements (e.g., a promoter, an enhancer, a repressor, etc.) one or more plasmids or plasmid fragments, one or more artificial chromosomes or fragments, or a combination thereof. A feature can be automatically selected. A feature can be user-selectable.

A “disease gene model” can refer to the mode of inheritance for a phenotype. A single gene disorder can be autosomal dominant, autosomal recessive, X-linked dominant, X-linked recessive, P-linked, or mitochondrial. Diseases can also be multifactorial and/or polygenic or complex, involving more than one variant or damaged gene.

“Pedigree” can refer to lineage or genealogical descent of an individual. Pedigree information can include polynucleotide sequence data from a known relative of an individual such as a child, a sibling, a parent, an aunt or uncle, a grandparent, etc.

A biological sample may comprise a sample from a subject, such as whole blood; blood products; red blood cells; white blood cells; buffy coat; swabs; urine; sputum; saliva; semen; lymphatic fluid; amniotic fluid; cerebrospinal fluid; peritoneal effusions; pleural effusions; biopsy samples; fluid from cysts; synovial fluid; vitreous humor; aqueous humor; bursa fluid; eye washes; eye aspirates; plasma; serum; pulmonary lavage; lung aspirates; animal, including human, tissues, including but not limited to, liver, spleen, kidney, lung, intestine, brain, heart, muscle, pancreas, cell cultures, as well as lysates, extracts, or materials and fractions obtained from the samples described above or any cells and microorganisms and viruses that may be present on or in a sample. A sample may comprise cells of a primary culture or a cell line. Tissues, cells, and their progeny of a biological entity obtained in vivo or cultured in vitro are also encompassed.

“Amino acid” or “peptide” refers to one of the twenty biologically occurring amino acids and to synthetic amino acids, including D/L optical isomers. Amino acids can be classified based upon the properties of their side chains as weakly acidic, weakly basic, hydrophilic, or hydrophobic. A “polypeptide” refers to a molecule formed by a sequence of two or more amino acids. Proteins are linear polypeptide chains composed of amino acid building blocks. The linear polypeptide sequence provides only a small part of the structural information that is important to the biochemist, however. The polypeptide chain folds to give secondary structural units (most commonly alpha helices and beta strands). Secondary structural units can then fold to give supersecondary structures (for example, beta sheets) and a tertiary structure. Most of the behaviors of a protein are determined by its secondary and tertiary structure, including those that are important for allowing the protein to function in a living system.

The term “alignment,” as used herein, generally refers to the arrangement of sequence reads to reconstruct a longer region of the genome. Reads can be used to reconstruct chromosomal regions, whole chromosomes, or the whole genome. The methods disclosed herein can be used to identify, rank, and score variants by relevance either individually or in sets lying within a feature. A feature can be any span or a collection of spans on the genome sequence or transcriptome sequences such as a gene, transcript, promoter, splice site, exon, intron, UTRs, genetic locus or extended gene region including regulatory elements. A feature can also be a list of 2 or more genes, a genetic pathway or an ontology category.

Disclosed herein is a newly developed analysis method that can analyze personal genome sequence data. The input of the method can be a genome file. The genome file can comprise genome sequence files, partial genome sequence files, genome variant files (e.g., VCF files, GVF files, etc.), partial genome variant files, genotyping array files, or any other DNA variant files. The genome variant files can contain the variants or difference of an individual genome or a set of genomes compared to a reference genome (e.g., human reference assembly). These variant files can include variants such as single nucleotide variants (SNVs), single nucleotide polymorphisms (SNPs), small and larger insertion and deletions (INDELS), rearrangements, CNV (copy number variants), Structural Variants (SVs), etc. The variant file can include frequency information for each variant.

The methods disclosed herein can be implemented as computer executable instructions or tools. In an embodiment, VAAST is such a tool. VAAST can provide a simple approach to accomplish these methods, allowing users to, for example:

(1) Combine and compare whole genome variant data for diverse downstream analyses;

(2) Identify hotspots of variation within a genome and its annotations, e.g., genes, regulatory elements, etc; and

(3) Perform ontology-driven analyses in order to investigate variation within a given Gene Ontology (GO) category, disease class or metabolic pathway.

In an embodiment, VAAST comprises an FPT (Feature Prioritization Tool). The FPT can be a general method to score variants in personal genomes for importance. In some embodiments, VAAST:

(1) Prioritizes features and/or variants;

(2) Can prioritize coding, non-coding and features composed of both, e.g., genes;

(3) Assign VAAST score to variants and/or features;

(4) Can leverage pedigree information;

(5) Can train itself on the fly; and

(6) Can use PG seqs, exomes, chip data, and datasets that are mixtures of all three.

In the context of human genome sequences, it can be applied as:

(1) a general probabilistic ‘disease-gene’ finder: identify disease-causing genes & their variants in genomes and sets of genomes;

(2) a search tool to rapidly search personal genome sequences for genes having significant differences in variant frequencies vs. controls;

(3) an approach to determine the statistical significance of ‘hits;’ and

(4) an approach to automatically relate hits to genetic pathways, network and ontologies.

These analyses can be carried out on sets of genomes, making possible both pairwise (single against single genome, single against set of background genomes) and case-control style studies (set(s) of target genomes against set of background genomes) of personal genome sequences. The present disclosure provides several analyses of healthy and cancer genomes and shows how VAAST can be used to identify variation hotspots both along the chromosome, and within gene ontologies, disease classes and metabolic pathways. Special emphasis is placed upon the impact of data quality and ethnicity, and their consequences for further downstream analyses. The present disclosure also shows how variant calling procedures, pseudogenes and gene families can all combine to complicate clinically-orientated analyses of personal genome sequences in ways that only become apparent when cohorts of genomes are analyzed.

The FPT search method in VAAST can be applied to identify disease genes in novel genome sequences such as a whole human genome variant file. It can automatically prioritize features such as genes in a genome sequence based on the relevance of variants within the gene. A typical example is an individual with a rare genetic disease for which a researcher or doctor seeks to identify the causative variant(s)/mutation(s). The recently published Miller Syndrome analysis given an exome sequence provides a test case for VAAST: Nat Genet. 2010 January; 42(1):30-5. Epub 2009 Nov. 13; and whole genome sequencing: Nat Methods. 2010 May; 7(5):350.). Given a variant file of 4 million single nucleotide variants (SNVs) the two disease causing genes where ranked 3rd and 11th given only the two affected personal genomes, and using dbSNP and data from the 1000 Genomes project as the set of background genomes. When pedigree information is included—for example, both parents—VAAST FPT assigns a statistically significant score to the two disease genes in question, and they are ranked on top (see Example 7).

The FPT search method in VAAST can be applied to case/control like studies (such as GWAS-like genome sequence studies). It can automatically rank which features/genes are most likely be disease relevant in the patient genomes versus the healthy genomes.

The FPT search method in VAAST can be applied to pairwise studies such as cancer analyses, where studies have sets of pairs of tumor and germline genomes from patients (often from skin). The task is to identify features that are relevant in tumor development (often somatic mutations) in the target (tumor) genomes versus background (germline) genomes.

The FPT search method in VAAST can be applied to progression studies in genomes such as different stages in tumor development (solid tumor, metastasis, recurrence, etc.).

The FPT search method in VAAST can be applied to genomes to remove noise of systematic errors in sequencing technologies. If a sequencing technology has systematic sequencing difficulties for a specific genome position or area, analyses of sets of genomes from the same technology can automatically filter out these errors during the VAAST analysis.

The FPT search method in VAAST can be applied to identify sites of purifying selection or rapid evolution in novel and agricultural genomes.

In addition to the likelihood ratio test as a scoring mode, VAAST can incorporate several alternative feature-ranking algorithms; for example, the Weighted Sum Statistic (wss) method (Madsen and Browning, PLoS Genet. 2009 February; 5(2): e1000384; which is hereby incorporated by reference in its entirety) for ranking features.

### Online Mendelian Inheritance in Man (OMIM)

Online Mendelian Inheritance in Man (OMIM) is a database that catalogues all the known diseases with a genetic component, and—when possible—links them to the relevant genes in the human genome and provides references for further research and tools for genomic analysis of a catalogued gene (see www.omim.org). OMIM is one of the databases housed in the U.S. National Center for Biotechnology Information (NCBI) and included in its search menus. Every disease and gene is assigned a six digit number of which the first number classifies the method of inheritance. If the initial digit is 1, the trait is deemed autosomal dominant; if 2, autosomal recessive; if 3, X-linked. Wherever a trait defined in this dictionary has a MIM number, the number from the 12th edition of MIM, is given in square brackets with or without an asterisk (asterisks indicate that the mode of inheritance is known; a number symbol (#) before an entry number indicates that the phenotype can be caused by mutation in any of two or more genes) as appropriate (e.g., Pelizaeus-Merzbacher disease [MIM #312080] is an X-linked recessive disorder).

Range of MIM codes for method of inheritance: 100000-299999: autosomal loci or phenotypes (created before May 15, 1994); 300000-399999: X-linked loci or phenotypes; 400000-499999: Y-linked loci or phenotypes; 500000-599999: Mitochondrial loci or phenotypes; 600000-above: Autosomal loci or phenotypes (created after May 15, 1994). Allelic variants (mutations) are designated by the MIM number of the entry, followed by a decimal point and a unique 4-digit variant number. For example, allelic variants in the factor IX gene (300746) are numbered 300746.0001 through 300746.0101.

In an embodiment, the method of identifying and/or prioritizing phenotype causing variants by comparing a target cohort to a background cohort incorporates Amino Acid Substitution (AAS) information from the OMIM database.

### BLOSUM Substitution Matrixes

BLOSUM substitution matrixes can be used to align protein sequences. They are based on local alignments. A BLOSUM substitution matrix contains the calculated log-odds (LOD) score for each of the 210 possible substitutions of the 20 standard amino acids. All BLOSUM matrices are based on observed alignments. Several sets of BLOSUM matrices exist using different alignment databases, named with numbers. BLOSUM matrices with high numbers are designed for comparing closely related sequences, while those with low numbers are designed for comparing distant related sequences. For example, BLOSUM80 is used for less divergent alignments, and BLOSUM45 is used for more divergent alignments. The matrices can be created by merging (clustering) all sequences that can be more similar than a given percentage into one single sequence and then comparing those sequences (that can all be more divergent than the given percentage value) only; thus reducing the contribution of closely related sequences. The percentage used can be appended to the name, giving BLOSUM80 for example where sequences that can be more than 80% identical are clustered.

Scores within a BLOSUM are log-odds scores that measure, in an alignment, the logarithm for the ratio of the likelihood of two amino acids appearing with a biological sense and the likelihood of the same amino acids appearing by chance. The matrices are based on the minimum percentage identity of the aligned protein sequence used in calculating them. Every possible identity or substitution is assigned a score based on its observed frequencies in the alignment of related proteins. A positive score is given to the more likely substitutions while a negative score is given to the less likely substitutions.

To calculate a BLOSUM matrix, the following equation is used: Sᵢⱼ=(1/λ) log [pᵢⱼ/(qᵢ*qⱼ)], where pᵢⱼ is the probability of two amino acids i and j replacing each other in a homologous sequence, and qᵢ and qⱼ are the background probabilities of finding the amino acids i and j in any protein sequence at random. The factor λ is a scaling factor, set such that the matrix contains easily computable integer values.

In an embodiment, the method of identifying and/or prioritizing phenotype causing variants by comparing a target cohort to a background cohort incorporates Amino Acid Substitution (AAS) information from a BLOSUM substitution matrix. The BLOSUM substitution matrix can be any BLOSUM substitution matrix; for example, BLOSUM30, BLOSUM31, BLOSUM32, BLOSUM33, BLOSUM34, BLOSUM35, BLOSUM36, BLOSUM37, BLOSUM38, BLOSUM39, BLOSUM40, BLOSUM41, BLOSUM42, BLOSUM43, BLOSUM44, BLOSUM45, BLOSUM46, BLOSUM47, BLOSUM48, BLOSUM49, BLOSUM50, BLOSUM51, BLOSUM52, BLOSUM53, BLOSUM54, BLOSUM55, BLOSUM56, BLOSUM57, BLOSUM58, BLOSUM59, BLOSUM60, BLOSUM61, BLOSUM62, BLOSUM63, BLOSUM64, BLOSUM65, BLOSUM66, BLOSUM67, BLOSUM68, BLOSUM69, BLOSUM70, BLOSUM71, BLOSUM72, BLOSUM73, BLOSUM74, BLOSUM75, BLOSUM76, BLOSUM77, BLOSUM78, BLOSUM79, BLOSUM80, BLOSUM81, BLOSUM82, BLOSUM83, BLOSUM84, BLOSUM85, BLOSUM86, BLOSUM87, BLOSUM88, BLOSUM89, BLOSUM90, BLOSUM91, BLOSUM92, BLOSUM93, BLOSUM94, BLOSUM95, BLOSUM96, BLOSUM97, BLOSUM98, BLOSUM99, or BLOSUM100. In an embodiment, the BLOSUM substitution matrix is BLOSUM 62.

### Computer Systems

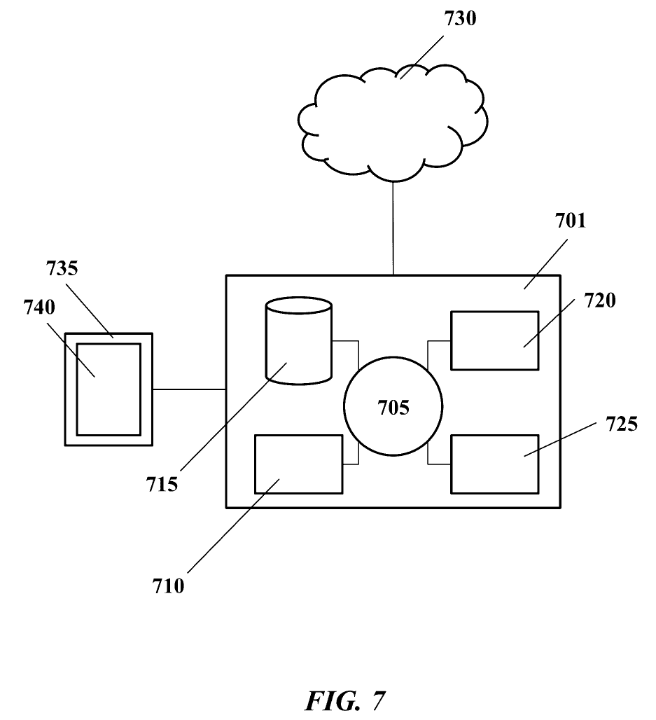

The present disclosure provides computer control systems that are programmed to implement methods of the disclosure. FIG. 7 shows a computer system 701 that is programmed or otherwise configured to implements methods of the present disclosure. The computer system 701 can be integral to implementing methods provided herein, which would be otherwise extremely difficult to perform in the absence of the computer system 701. The computer system 701 can regulate various aspects of methods of the present disclosure, such as, for example, methods that integrate phenotype and disease information with personal genomic data for improved power to identify disease-causing alleles (pVAAST). The computer system 701 can be an electronic device of a user or a computer system that is remotely located with respect to the electronic device. The electronic device can be a mobile electronic device. As an alternative, the computer system 701 can be a computer server.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet. The network 730 in some cases is a telecommunication and/or data network. The network 730 can include one or more computer servers, which can enable distributed computing, such as cloud computing. The network 730, in some cases with the aid of the computer system 701, can implement a peer-to-peer network, which may enable devices coupled to the computer system 701 to behave as a client or a server.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage unit 715 can store files, such as drivers, libraries and saved programs. The storage unit 715 can store user data, e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user (e.g., patient, healthcare provider, or service provider). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The memory 710 can be part of a database. The machine executable or machine readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 can include or be in communication with an electronic display 735 that comprises a user interface (UI) 740 for providing, for example, genetic information, such as an identification of disease-causing alleles in single individuals or groups of individuals. Examples of UI's include, without limitation, a graphical user interface (GUI) and web-based user interface (or web interface).

pVAAST

The Variant Annotation, Analysis and Search Tool (VAAST) can employ a variant association test that combines amino acid substitution and allele frequency information using a composite likelihood ratio test (CLRT). In an embodiment provided herein the VAAST methodology is augmented by incorporating a pedigree-based tool, pedigree-VAAST (pVAAST), based on VAAST and incorporates family data. pVAAST performs linkage analysis by calculating a gene-based LOD score using a model specifically designed for sequence data with support for dominant, recessive, and de novo inheritance. This model is capable of modeling de novo mutations and is more sensitive in scenarios with incomplete penetrance or locus heterogeneity. The LOD score at each locus is incorporated directly into the CLRT to increase the accuracy and greatly decrease the technical complexity of family-based disease-gene identification efforts. Statistical significance can be calculated using a combination of permutation and gene-drop simulation¹ to account for both the family structure and the observed pattern of variation in cases and controls. This tool can be applied to whole-genome data from a 5-generation pedigree with cardiac septal defects in 11 of 21 sequenced family members. A missense mutation in the gene GATA4 had been previously identified as the variant responsible for cardiac septal defects in the family. In a whole-genome analysis, GATA4 may be highly significant (p=3×10⁻⁹), and no other gene may reach genome-wide significance (p<2.5×10⁻⁶). Further, the genotypes and phenotypes in the pedigree can be modified to demonstrate that pVAAST is robust to incomplete penetrance, locus-heterogeneity, and ambiguous phenotyping. pVAAST maintains high power across a wide variety of study designs, from monogenic, Mendelian diseases in a single family to highly polygenic, common diseases involving hundreds of families.

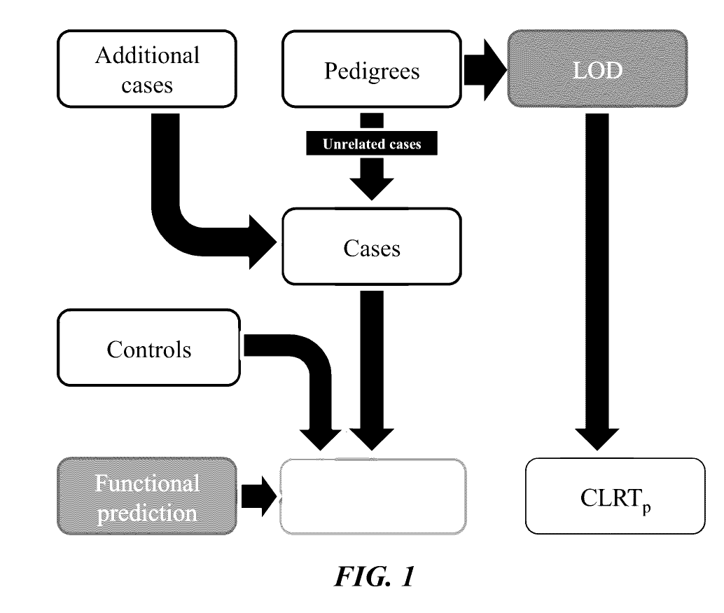

Pedigree VAAST (pVAAST), an algorithm that incorporates both linkage analysis and statistical association in a unified statistical framework. Unlike LOD scores in traditional parametric linkage analysis, the LOD score in pVAAST is designed for sequence data. Specifically, the statistical model assumes that the functional variants influencing disease-susceptibility can be directly detected. This assumption allows pVAAST to calculate LOD score for de novo mutations, which may not be possible with traditional linkage analysis. pVAAST is built upon the CLRT used in VAAST, but in addition integrates the linkage information (quantified by a LOD score) as a separate log likelihood ratio in the pVAAST CLRT (CLRTₚ) (FIG. 1). pVAAST evaluates the significance of the CLRTₚ score using permutation and gene-drop simulation¹. Here, the utility of pVAAST in a variety of simulated and real datasets across a broad range of family-based study designs is demonstrated.

### EXAMPLES

### Example 1. Basic LOD Score Calculation in pVAAST

In classic two-point parametric linkage analysis, the marker under investigation is usually assumed not to be causal, but rather linked with the actual causal variant with a certain recombination probability (r). Under the null hypothesis, r=0.5, which indicates that the marker and causal mutation are unlinked. Under the alternative hypothesis, r is a free parameter. Given the disease prevalence, allele frequency of the marker and causal allele, and the penetrance of the causal allele, the likelihood of alternative and null model can be calculated for given values of r using Elston-Stewart algorithm¹⁸. The log 10 ratio of the maximum likelihood of the alternative and null model is the LOD score.

pVAAST is designed for sequencing data and thus assumes that the causal mutation can be directly observed. For simplicity, the term causal can refer to any variant that directly increases disease risk, regardless of penetrance. As a result, an alternative model can be used to calculate the LOD score. It can be assumed that the disease is caused by either the locus under investigation (current locus), or some other unlinked locus in the genome (latent locus). In both models, the current and latent loci are unlinked and there is no epistatic interaction between the alleles. The null model states that variant(s) in the latent locus cause the disease with some probability, and the current locus is not causal. The alternative model states that variants in both the current and latent loci can independently cause the disease, with different probabilities. Intuitively, the null model attributes the disease phenotype solely to the latent locus, and the alternative model allows variants in both the current and latent loci to be independently causal. The likelihoods of the alternative and null models can then be maximized over ρ_{c} (genotype disease probability vector for the current variant), ρₗ (genotype disease probability vector for the latent locus) and fₗ (minor allele frequency of the latent locus) and calculate the log 10 likelihood ratio as the LOD score. Formally,

LOD=log₁₀ max L(alt)−log₁₀ max L(null), (Equation 1)

while the likelihood for both null model and alternative model has the form:

L=P(g_{c},gₗ,p|ρ_{c},ρₗ,f_{c},fₗ). (Equation 2)

Here g_{c} and gₗ are the genotype vectors (with values of 0, 1 and 2 corresponding to homozygous-reference, heterozygous and homozygous non-reference genotypes) of the current and latent variant sites; p is the phenotype vector of the pedigree, and f_{c} and fₗ are the allele frequencies of the current and latent alleles. For the null model, the expression can be further decomposed into

Pₙᵤₗₗ(g_{c},gₗ,p|ρ_{c},ρₗ,f_{c},fₗ)=P(g_{c}|f_{c})P(gₗ,p|ρₗ,fₗ). (Equation 3)

because only the latent allele is causal for the disease under the null model, and is thus independent from p, gₗ and ρₗ.

Given ρ_{c}, ρₗ, f_{c}, and fₗ, all of the aforementioned probabilities can be calculated with the Elston-Stewart algorithm¹⁹ in linear computational time relative to the family size. The parameter f_{c} can be estimated from the allele frequency in a control population, and perform a grid search over ρ_{c}, ρₗ and fₗ to maximize the likelihood. For the dominant model, a heterozygous genotype is sufficient to be considered as a risk genotype; for a simple recessive model, a homozygous genotype is required (with the exception of sex chromosomes). Compound heterozygous scenarios are discussed in the next section.

If more than one family is present, the LOD score is calculated for each family separately but requires ρ_{c} to be consistent across families. Then, within each family, the LOD of one variant is chosen to be the gene LOD score of this family. By default, the variant with the highest CLRTᵥ score is chosen, but the user can opt to use CLRTₚ score or LOD score alone as well. In practice, in large pedigrees using the CLRTᵥ score as a selection criterion may yield more favorable results. Finally, the sum of the gene LOD score from multiple families is used to generate the overall pVAAST LOD score.

### Example 2. Extension to Compound Heterozygous and De Novo Disease Models

The compound heterozygous recessive disease model requires special attention because the genotype vectors (gₗ and g_{c}) now involve more than one variant site. To illustrate, consider a gene with three polymorphism sites i, j and k. A natural approach to calculate the gene LOD score can be to calculate the LOD for all pairwise combination of heterozygous variant sites within the gene (i.e, i+j, i+k, and i+k) separately and then select the highest LOD score. This requires the evaluation of (n choose 2) combinations, where n is the number of variant sites in the gene. However, this approach is flawed because it assumes the genotype disease probabilities for all pairs of sites are independent, which is incorrect. Instead, it can be assumed that any variant in the gene is either causal (D-variants) or neutral (N-variants)²⁰ with the same relative risk. For example, if a gene has four heterozygous sites j, k and l, within which i, j, and k are causal, then an individual with at least two mutations occurring at i, j or k sites, or any combination thereof, on different two chromosomes can be at risk; otherwise she will not be at risk.

Under this model, a Boolean risk vector can be constructed for a gene to denote whether each variant within the gene is a D-variant or N-variant. If the underlying risk vector for some gene is known, then the genotype of an individual can be determined by evaluating whether he or she carries at least one D-variant on each chromosome. Then the calculation of LOD score is reduced to the simple recessive case described above. However, finding the optimal risk vector is not a trivial problem, since a brute-force approach to find the risk vector maximizing the LOD score has a complexity of O(2̂n), where n is the number sites in the gene. To make this more efficient, a Markov chain Monte Carlo (MCMC) method can be used to approximate the optimal risk vector. Briefly, given a particular risk vector and the phenotype probability for each genotype, the joint likelihood for all sequenced and phenotyped individuals can be calculated as

L=ρᵣⁿ^{ₐ}(1−ρᵣ)ⁿ^{_{b}}ρₙⁿ^{_{c}}(1−ρₙ)ⁿ^{_{d}}, (Equation 4)

where ρᵣ is the probability that an individual with a risky genotype is affected; ρₙ is the probability that an individual with a neutral genotype is affected; nₐ and n_{b} are the total numbers of affected/unaffected individuals with risky genotype; n_{c} and n_{d} are the total number of affected/unaffected individuals with neutral genotypes. Both ρᵣ and ρₙ are configurable parameters, although the performance of the MCMC method is usually insensitive to these parameters.

Starting with a random risk vector, a variant site is randomly selected to switch to the opposite value (neutral to risky and risky to neutral). The likelihoods for both risk vectors are calculated and the new risk vector is selectively accepted according to the Metropolis-Hastings method. This process is repeated until convergence or the maximal number of iterations is achieved. Lastly, the most likely risk vector is selected from the Markov Chain and the LOD score calculation is performed as described in the previous section.

In some cases, de novo mutations can be accommodated by allowing Mendelian inheritance errors to occur in the pedigree likelihood calculation. Specifically, in the Elston-Stewart algorithm¹⁸, if the offspring carries a mutation absent from both parents, then this transmission has a probability of m (mutation rate per site per generation in human genome; default 1.2e^{-8 12}). Accordingly, Mendelian inheritance error is randomly introduced in the gene-drop simulations' with a probability equal to the genotyping error rate.

### Example 3. Integrating LOD Scores into Composite Likelihood Ratio (CLR) Test

pVAAST is built upon the framework of Variant Annotation Analysis and Search Tool (VAAST)⁶. VAAST uses an extended composite likelihood ratio test (CLRT) to determine a severity score for genomic variants. The null model of the CLRT states that the frequency of a variant or variant group is the same in the control population (background genomes) and case population (target genomes), while the alternative model allows these two frequencies to differ. Under a binomial distribution, the likelihood for both models can be calculated based on observed allele frequencies in the control and case datasets. This likelihood is further updated by calibrated amino acid substitution and indel severity weights.

To integrate genetic linkage information into the CLRT, one sequenced and affected individual from each pedigree (pedigree representative) is selected to establish a group of cases. The IDs of the selected individuals can be provided, but if such information is absent, pVAAST will randomly choose one individual carrying the highest-scoring variant in the current gene. Additional affected individuals not present in any family can also be included among the cases. The natural log of composite likelihood ratio, λ, is calculated as previously described⁶, then the LOD score term is added in to calculate the pVAAST CLRT (CLRTₚ) score:

$${λ}^{′} = c ⋆ ( {\sum }_{i = 1}^{n} {LOD}_{{m}_{i}} ) + λ , \qquad ( Equation 5 )$$

where

c=2*ln(10). (Equation 6)

To avoid confusion, the original CLRT score in VAAST (without the linkage component) is denoted as CLRTᵥ throughout this disclosure. FIG. 1 provides a schematic diagram for the calculation of the CLRTₚ scores in pVAAST. A combined approach of permutation and gene-drop simulation is used to evaluate the significance of the above test statistics. Specifically, for each iteration:

(1) One affected, sequenced individual is selected from each pedigree. These selected genomes are pooled with sporadic patient genomes as well as unrelated control genomes, denoted as chromosome Set A. Because pVAAST currently does not support phased genotype inputs, heterozygous variants are randomly phased into either chromosome copy on diploid chromosomes (autosome and X-chromosome in females). Inflation of Type I error as a result of the random-phasing procedure is not observed.

(2) To generate a random pedigree in the simulation, chromosomes from Set A are sampled, without replacement, as the pedigree founders. Within each nuclear family, the genotypes of the offspring are simulated by randomly sampling the chromosomes from the parents according to Mendelian inheritance. This process is repeated until the genotypes of all pedigree members are simulated.

(3) If additional unrelated cases are included in the analysis, then these genomes are also sampled (without replacement) from Set A. The remaining genomes (those not selected) in set A are used as the control genomes.

(4) The combined CLRTₚ test statistics are calculated as before, based on the simulated genome set.

Note since each pedigree contributes only one genome into Set A, but usually uses more than two chromosomes in the gene-drop, the number of control genomes in the permutation is usually smaller than actual number of control genomes provided by the user. To compensate for this, a fixed number of randomly selected control genomes can be removed from the real scenario, so that the control sample size matches that in the permutation. This process is repeated n times (100 by default); each time a set of control genomes for removal is sampled and the corresponding CLRTₚ score is calculated. Thus, the CLRTₚ test statistic in the real scenario is actually a vector of length n. In the simulation, the CLRTₚ score is compared to a CLRTₚ score randomly sampled from the n-vector. Finally, after all the iterations are finished, the p-value is calculated by:

p=(s+l)/(t+l), (Equation 7)

where t is the total number of iterations performed, and s is the number of iterations where the test statistics is no less than the real-scenario test statistic.

### Example 4. Control Genomes for Cardiac Septal Defect Pedigree and Miller Syndrome Pedigrees

The control genome set consists of 1057 exomes from 1000 Genomes Project Phase I data²¹, 54 genomes from Complete Genomics Diversity Panel²², 184 Danish exomes²³, and 9 non-duplicative genomes from the 10Gen dataset²⁴, representing a wide variety of ethnicities and sequencing platforms. In order to include a wider set of variants that are unlikely causal for rare Mendelian diseases, high-quality variants (defined as polymorphism sites with sample size no less than 100 chromosomes) are also collected from dbSNP build 137²⁵ and NHLBI exome sequencing data²⁶. These variants are then randomly inserted into the control genome set, setting the minor allele frequency equal to the reported value. This genome set is used as controls for the results presented in Examples 11-13.

### Example 5. pVAAST Parameterization

pVAAST uses a configuration file (.ctl) to parameterize each analysis. In addition to the standard configuration template, a template is also provided for analyzing relatively small-size pedigrees with rare Mendelian diseases by modifying the following parameters: 1) ‘lod_score_filter:yes’, which removes genes without a positive LOD-score from further permutation; 2) ‘nocall_filter: yes’, which filters polymorphism sites with more than one missing genotypes in sequenced individuals; 3) ‘inheritance_error_filter: yes’, which filters polymorphisms sites containing an inheritance error; 4) ‘penetrance_lower_bound: 0.995’, which sets the minimal penetrance of the risk variant to close to 100%. When the sample size is small, these stringent filtering criteria improve the statistical power of detecting causal genes for rare Mendelian diseases with high penetrance.

In a pVAAST configuration file, two options often require attention: ‘unknown_representatives’ and ‘max_prevalence_filter’. Since pVAAST selects one affected individual from among the sequenced individuals in each pedigree as the pedigree representative to calculate CLRTᵥ score, the user can be required to designate the IDs of the pedigree representatives. If the designated individual is a phenocopy, pVAAST will miss the true causal variant. To compensate for this behavior, the user can set ‘unknown_representatives” to yes to instruct pVAAST to automatically select the pedigree representative that maximizes CLRTₚ. This procedure makes the p-value more conservative, but significantly reduces false-negative rates in large pedigrees. The ‘max_prevalence_filter’ specifies the disease prevalence and restrict the grid search algorithm in LOD score calculation to parameter space permitted by the designated prevalence. It is suggested that users set this parameter to the upper bound of estimated disease prevalence, but if such information is unavailable or controversial, a conservative approach is to use the default value (0.1) in the configuration template file.

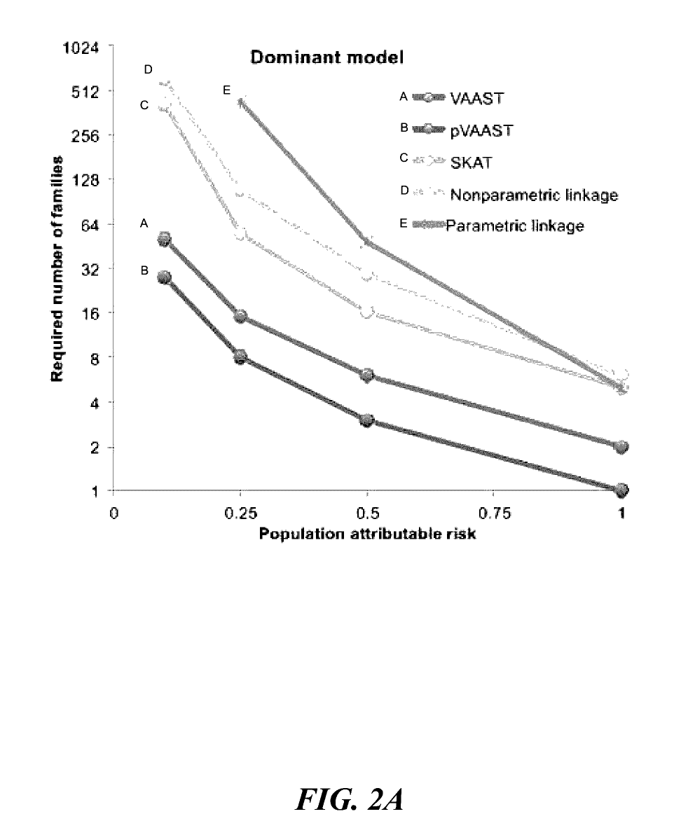

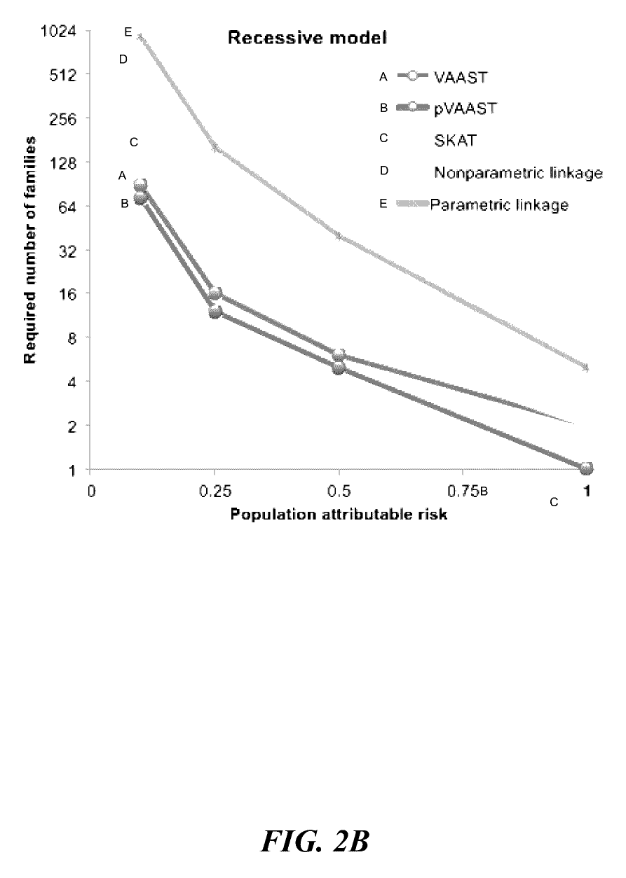

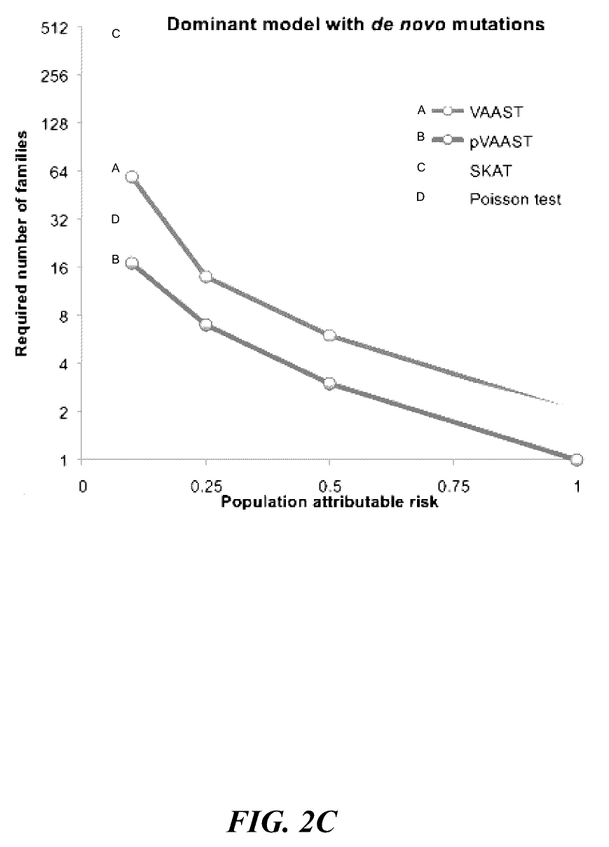

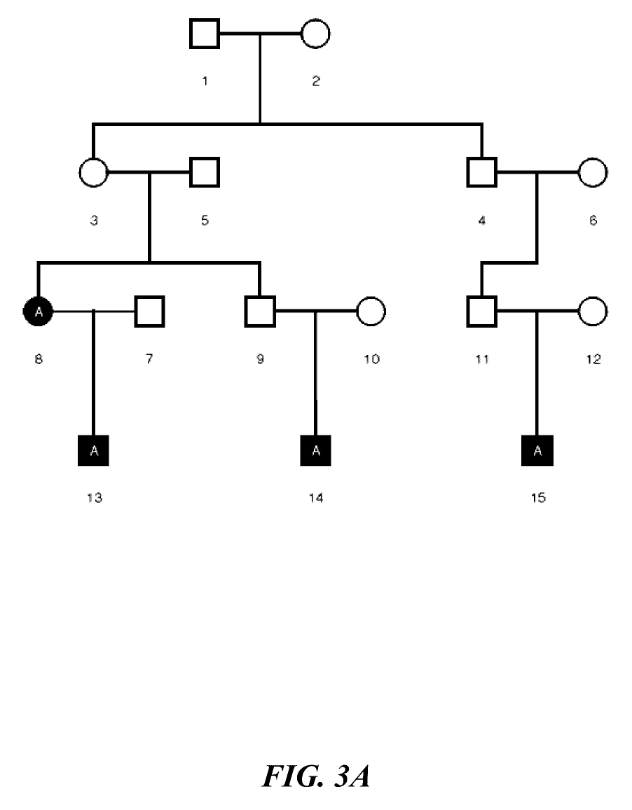

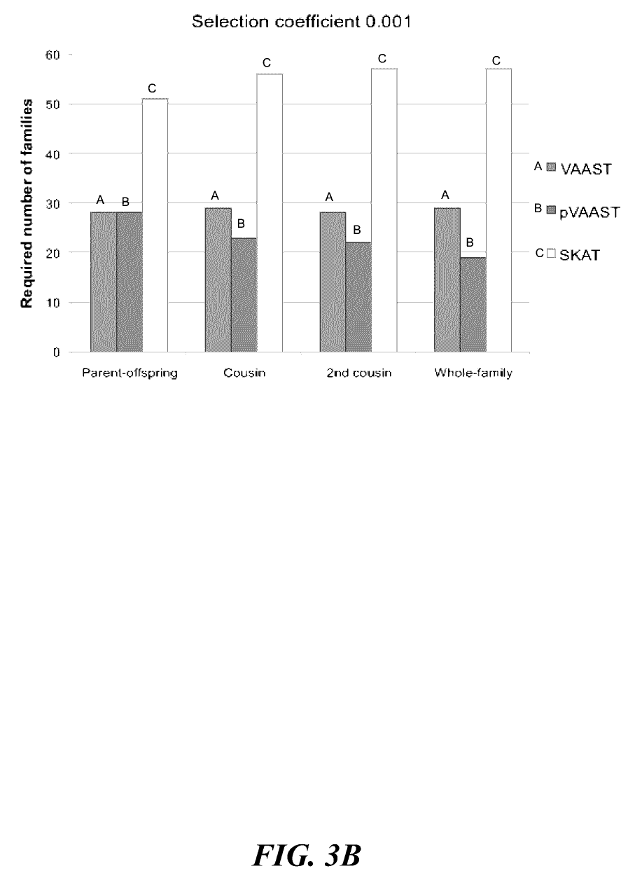

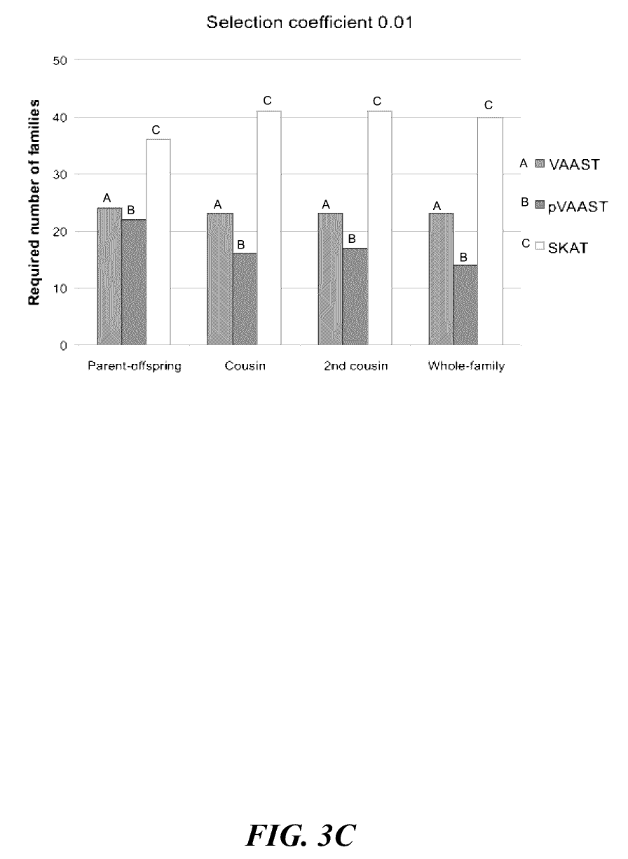

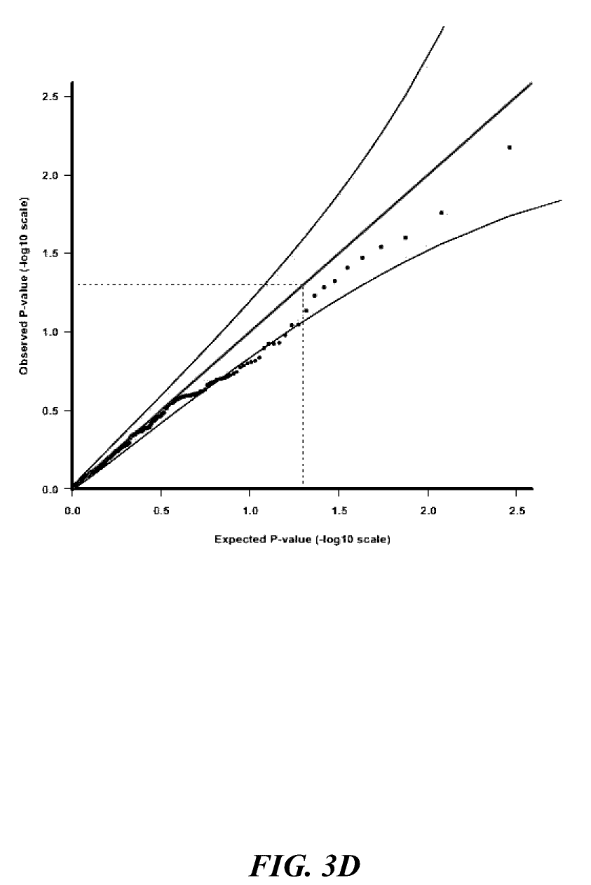

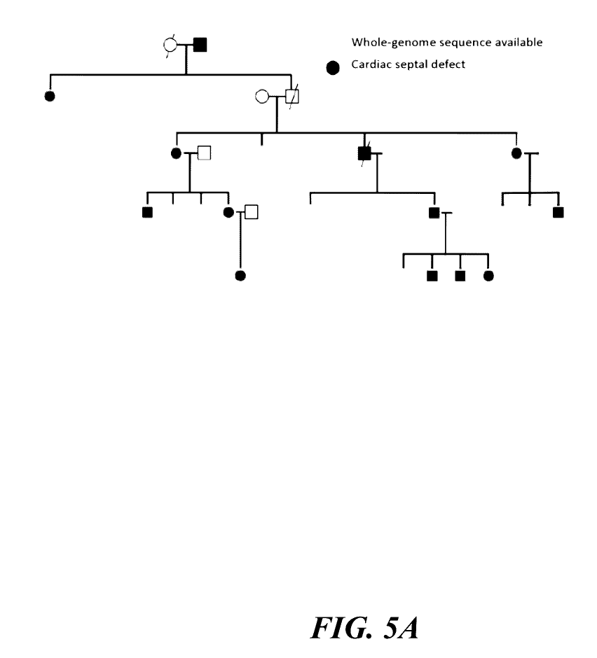

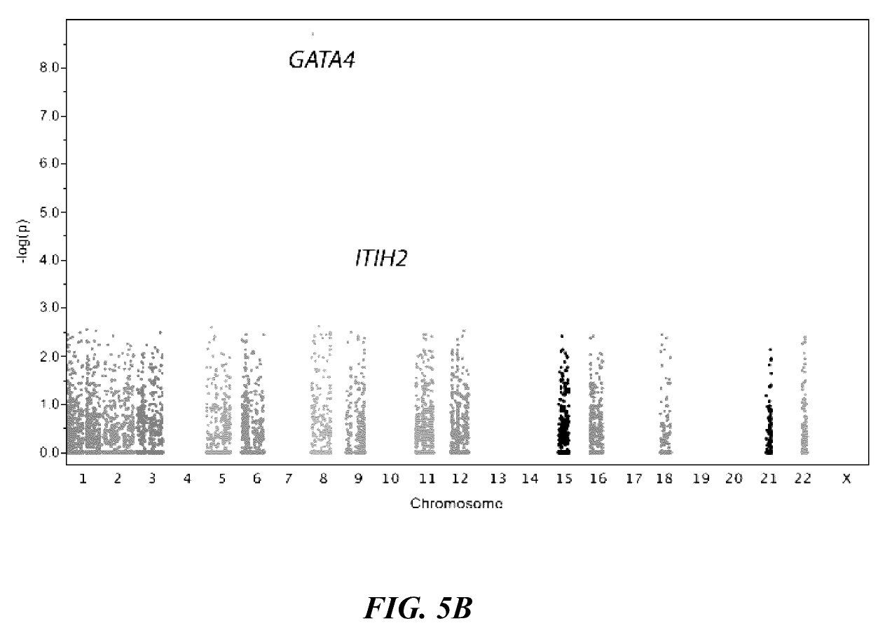

In Example 7 and Example 12, the small-pedigree rare Mendelian disease template file is used. For all other examples, the standard configuration template is used. The recessive and dominant simulations in FIG. 2 and FIG. 3 did not incorporate genotype error, and thus he “simulate genotyping error” option for these analyses is disabled. In all analyses involving the cardiac septal defect pedigree (FIG. 5), ‘unknown_representatives’ option is used as discussed previously. The ‘max_prevalence_filter’ option is set to an estimate of the disease prevalence in each analysis (5e-5 for rare Mendelian disease simulations and Miller syndrome, 0.01 for the full cardiac septal defect pedigree, and 0.05 for common disease simulations). For the modified cardiac septal defects pedigree (FIG. 5), the choice of ‘max_prevalence_filter’ is often not obvious due to modified penetrance and locus-heterogeneity. For simplicity, this parameter is set to the default value of 0.1 throughout this analysis.

### Example 6. pVAAST Runtime

pVAAST supports multi-threading parallelization. The computational time is proportional to the size of pedigree and to the rounds of permutations being performed. On a linux server with Intel® Xeon® 2.00 GHz CPUs, the cardiac septal defect pedigree takes 181 hours (clock time) using 40 threads (maximum permutation: 1e9). The Miller syndrome pedigree takes 0.3 hours (clock time) using 70 threads (maximum permutation: 1e6).

### Example 7. Simulation in Rare Mendelian Diseases

To compare the relative power of VAAST, pVAAST, SKAT and non-parametric linkage analysis using Identity By Descent (IBD) information (see FIG. 2), pedigrees affected by a rare, Mendelian disease are simulated. Under the dominant simulation, a three-generation pedigree with two sequenced affected cousins is used. Under the recessive simulation, a two-generation pedigree with two sequenced affected siblings is used. The de novo simulation employes a two-generation pedigree with one affected child, and all three family members are sequenced. The dominant simulation presumes one risk variant is sufficient to cause the disease; the recessive simulation requires a pair of risk variants; the de novo simulation is the same as dominant but the causal variant is a de novo germline mutation. To explore the impact of locus-heterogeneity, four values of population attributable risks (PARs) are investigated: 10%, 25%, 50% and 100%. For example, a PAR of 10% indicates that among all simulated pedigrees, 10% carry the disease-causal variants in the gene under investigation, while the remaining 90% have risk variants elsewhere in the genome. Following²⁰, 50 different disease-causal alleles (SNVs) in the gene are allowed, so that affected individual may carry any one of the 50 causal variants. 50 risk-neutral alleles in the gene of interest whose alleles frequencies conform to Wright's formula of variant allele frequency²⁷ are also simulated. In the control population, neutral alleles have the same minor allele frequency as in affected cousin pairs, but no causal variants are present.

Phenotype information from the whole pedigree and sequencing data from the sequenced affected individuals is used as the input for pVAAST. To run VAAST and SKAT, one sequenced affected individual is selected from each pedigree to construct the case cohort, and the set of control genomes is the same as that used by pVAAST. The power is calculated with a non-parametric linkage method based on IBD in dominant and recessive simulations. Namely, the p-value is calculated for the observed number of sequenced affected pairs sharing Identity By Descent (IBD) at the investigated gene locus, under the null hypothesis the current locus is not linked to the disease locus. Specifically, the dominant null model tests whether the probability of two cousins sharing one chromosome is 1/4, and the recessive null model tests whether the probability of the siblings sharing both chromosomes is 1/4.

Under the de novo model, the power of association tests is also compared to a Poisson-based test for excess of inheritance errors. The rationale for this test is that germline inheritance errors (which can be caused by either germline mutations or genotyping errors) randomly occur in genome according to a Poisson process. Given the parameter of the Poisson distribution, the one-tail probability of the observed number of inheritance errors can be calculated, assuming these inheritance errors are not associated with the disease. In reality, the mean of the Poisson distribution is not known but can be estimated by considering factors such as local GC content and genomic repeat levels²⁸. This parameter is assumed to be known and the simulated parameter is used to calculate power of the Poisson-based then. By doing so, an upper limit estimate is provided for the power of such a test when the parameter is estimated.

### Example 8. Simulation in Common Complex Diseases

In this experiment shown in FIG. 3, it is assumed there are 50 risk-neutral SNVs and 50 risk variants within the gene. The allele frequencies of both sets of SNVs are simulated using Wright's formula, although the selection coefficient (s) of the two sets may differ. Specifically, in the risk-neutral set, s is always 0.001 (corresponding to variants under mild purifying selection in human); in the risk variant set, both 0.001 and 0.01 are explored as values for s (the latter corresponds to variants under strong purifying selection in human). For simplicity the population attributable risk (PAR) of all risk variants is assumed to be identical, and the odds ratio (r) can be calculated via following transformation:

$$r = \frac{α}{( 1 - α ) {q}_{U}} + 1 , \qquad ( Equation 8 )$$

consistent with²⁰. Here a stands for individual variant PAR and q_{U} stands for the allele frequency in control population. The total PAR and prevalence of the disease are both set at 0.05 to simulate a common genetic disease with a modest level of locus heterogeneity, such as breast cancer^{29,30}.

The affected pedigrees are assumed to all have the pedigree structure shown in FIG. 3A. Founder genotypes is simulated by randomly sampling alleles from aforementioned distributions, and the genotypes of remaining pedigree members are generated via gene-drop simulation′, assuming no recombination occurred. Rejection sampling is used to obtain pedigrees with the desired phenotypes. In each pVAAST run, one of the following sets of individuals is assumed to be sequenced: 1) trios (indi7, 8 and 13); 2) first cousin pairs (indi 13 and 14); 3) second cousin pairs (indi 13 and 15) or 4) entire pedigrees. In comparison, VAAST and SKAT runs include indi 13 from each family in the cohort cases.

### Example 9. Manipulating Congenital Heart Defect Pedigree to Create Test Case Under a Variety of Genetic Disease Settings

Different genetic disease setting can be simulated with the cardiac septal defect pedigree, using the following protocols:
To represent different levels of penetrance for the G296S mutation, affected pedigree members are randomly selected and their phenotypes are switched to unaffected without changing the genotype data. The original pedigree has 14 G296S mutation carriers. For example, to represent penetrance of 79%, the pedigree structure is modified so that 3 out 14 G296S carriers are unaffected.
To represent different levels of locus-heterogeneity, terminal G296S mutation carriers within the family tree are randomly selected and the G296S mutation is removed from GATA4.
To represent fewer number of informative meiosis, terminal nodes in the pedigree structure are randomly selected and removed from the pedigree, truncating the pedigree to a smaller size.
To represent ambiguous phenotypes, members in the pedigree are randomly selected, and their phenotypes are marked as “missing.”
   The following parameter sets are used for Superlink analysis:
Penetrance: ranges from 0 to 1 with increments of 0.1
Prevalence: 0.01
Recombination frequency: ranges from 0 to 1 with increments of 0.05

### Example 10. Simulated Family Data

The performance of pVAAST on rare Mendelian diseases is evaluated using simulated family data and unaffected control genomes. Three disease models are investigated using both association and pedigree-based approaches: dominant, recessive, and dominant resulting from de novo mutations. In all models, pVAAST is compared with two rare-variant association tests, VAAST^{6, 7} and SKAT⁴. For comparison, a non-parametric linkage method is included for dominant and recessive models. For the de novo model, a Poisson-based test is included, which detects excess inheritance error in cases. pVAAST correctly controls for Type I error in all three scenarios. FIG. 2 presents the required sample size by each method at four different levels of population attributable risk (PAR¹⁰). The required sample size of pVAAST is usually an order of magnitude lower than nonparametric linkage analysis, demonstrating the value of case-control sequencing data for rare Mendelian disease gene identification. Under dominant and de novo models, pVAAST typically requires half the sample size of VAAST, and one fifth the sample size of SKAT.

The performance of pVAAST is also benchmarked in common, complex diseases by simulating four-generation families (FIG. 3A). The relative performance of four different choices of sequenced pedigree members is compared: affected parent-offspring pairs, affected 1^{st} cousin pairs, affected 2^{nd} cousin pairs, and the entire pedigree. In FIG. 3B, risk alleles are mildly deleterious with a selection coefficient of 0.001, which resulted in an average MAF of 1.9×10⁻³. With all pedigree members in FIG. 3A, pVAAST requires only 66% of the sample size of VAAST, and with affected 1st cousin or 2^{nd} cousin pairs, pVAAST requires 79% the sample size of VAAST (FIG. 3B). With a selection coefficient of 0.01 (average MAF 2.2×10⁻⁴) (FIG. 3C), a similar trend is observed but with slightly better pVAAST performance in all scenarios. pVAAST correctly controls for Type I error in all scenarios (FIG. 3D). The performance of pVAAST is compared to ASKAT, an extension of SKAT that accommodates family-based studies. However, ASKAT controls for familial relationships through asymptotic assumptions, and for the relatively small sample sizes that are evaluated (up to 100 families), the Type I error of ASKAT is inflated.

### Example 11. Cardiac Septal Defect Pedigree

pVAAST is benchmarked on whole-genome sequencing data from a single pedigree affected with cardiac septal defects and having an autosomal dominant pattern of inheritance (FIG. 5A). Previously, Garg et al.¹¹ identified the G296S mutation in GATA4 as the cause of cardiac septal defects in this pedigree using genome-wide linkage mapping followed by sequencing of the GATA4 coding region and functional studies. pVAAST successfully identifies GATA4 with genome-wide significance (p=3.0×10⁻⁹; FIG. 5B). The G296S mutation has a CLRTᵥ score of 13.2 and a LOD score of 5.47, and no other variants receives a positive CLRTᵥ or LOD score in GATA4. The second ranking gene is ITIH2, with a p-value of 2.3e-5 and LOD score of 1.51. Because the prevalence parameter (disease prevalence in general population) is set to 0.01 to match that of cardiac septal defects, no other gene receives a positive LOD score in the pedigree. ASKAT is not applicable to this example due to the small sample size.

### Example 12. Miller Syndrome Pedigree

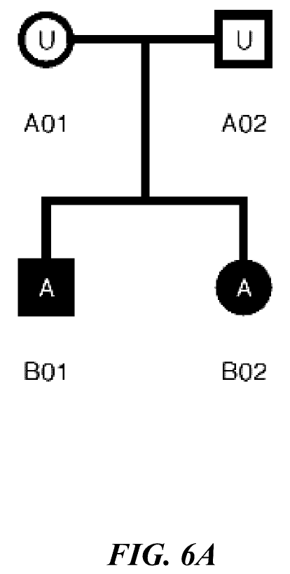

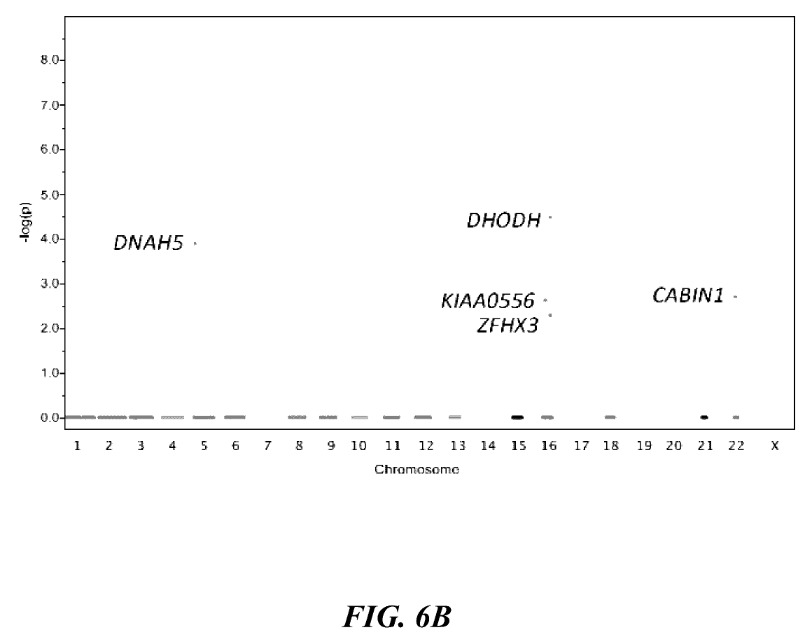

The performance of pVAAST on a recessive disease, Miller syndrome′², is investigated using whole-genome sequencing data from a two-generation pedigree (FIG. 6A). The two offspring are affected with Miller syndrome and primary ciliary dyskinesia, both of which are rare recessive Mendelian diseases. The two diseases are caused by compound heterozygous mutations in the DHODH and DNAH5 genes, respectivelyl². pVAAST identifies only 5 genes with positive LOD scores, and the two disease-causal genes (DHODH and DNAH5) are ranked as 1^{st} and 2^{nd} genome-wide (FIG. 6B), with p-values of 3.3×10⁻⁵ and 1.3×10⁻⁴, respectively. In both genes, only the two causal mutations receive positive scores, while all other variants have a score of zero.

### Example 13. pVAAST is Robust to Common Factors that Compromise the Power of Linkage Studies

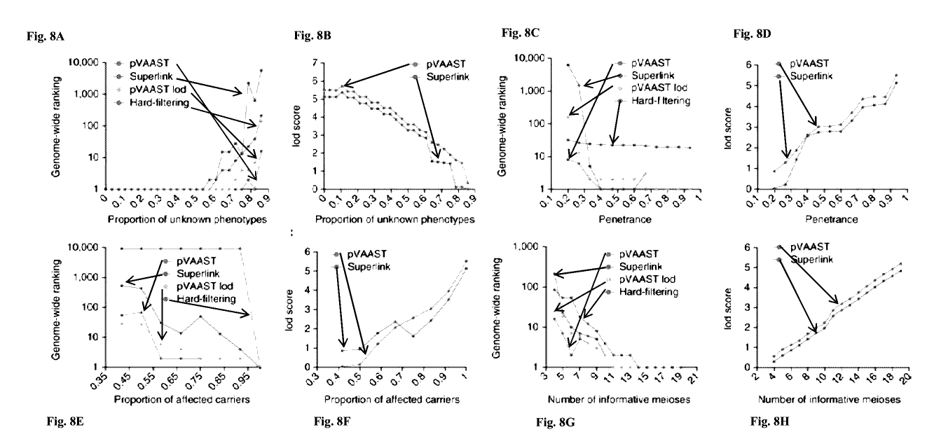

In linkage analysis, factors such as incomplete penetrance, locus heterogeneity, and ambiguous phenotyping negatively impact linkage signals and thus reduce disease-gene identification power. The cardiac septal defect pedigree data presented above (see Example 11 and FIG. 5) is a large pedigree with no locus heterogeneity and very high penetrance (93.3%) for the G296S mutation. The genotype and phenotype data from this pedigree can be modified to benchmark pVAAST in four scenarios involving 1) ambiguous phenotypes; 2) reduced penetrance; 3) locus heterogeneity; and 4) reduced number of informative meioses in the family. For each test case, the LOD score and the genome-wide ranking of GATA4 (ranked by p-values) are evaluated. The LOD score reported by pVAAST is approximately a monotonic function of each of the four parameters and is highly correlated with the classic two-point parametric LOD score (FIG. 8). pVAAST is robust to ambiguous phenotypes. For example, when 82% of pedigree members have unknown phenotypes, the LOD score of GATA4 is 1.5 and its genome-wide ranking is 1^{st} (FIG. 8A and FIG. 8B). Reduced penetrance generally decreased the LOD score without significantly compromising the genome-wide ranking (FIG. 8C and FIG. 8D). Specifically, the genome-wide ranking of GATA4 is consistently 1^{st} until the penetrance drops below 40%; even with penetrance of 20%, GATA4 is ranked 8^{th} genome-wide. In comparison, locus-heterogeneity has a greater impact on power (FIG. 8E and FIG. 8F). When locus heterogeneity is modest, GATA4 always ranks 1^{st} or 2^{nd}. However, when the proportion of affected individuals carrying G296S falls to 50%, the LOD score drops below 0.2 and the genome-wide ranking is beyond 50^{th}. The original family has 20 informative meiosis events, and results show pVAAST ranks GATA4 1^{st} genome-wide even when there are only 11 informative meioses in the family (FIG. 8G). Furthermore, with only 6 meioses, pVAAST still rank GATA4 2^{nd} genome-wide. This suggests that for a rare Mendelian disease with high penetrance and low locus heterogeneity within the family, the risk gene can often be identified among the top hits genome-wide using a typical three-generation pedigree.

For comparison, the genome-wide ranking of GATA4 is evaluated with two alternative approaches. In the first approach a two-point parametric LOD score at each polymorphism site is calculated, and the LOD score from the best-scoring site overlapping a protein-coding gene is designated as the gene LOD score. All genes are then ranked by the gene LOD scores. In the second approach, the same procedure is applied to the pVAAST LOD score to calculate the ranking (FIG. 8). It is found that the pVAAST LOD score is consistently more robust than the classic parametric LOD score in challenging scenarios such as low penetrance, high locus heterogeneity, small sample size, and large fraction of unknown phenotypes. The ranking of GATA4 gene with pVAAST LOD scores is usually one order of magnitude higher than with Superlink. This performance difference may not be surprising given that traditional two-point LOD scores test the hypothesis of disease linkage rather than disease causation and have been developed for sparse marker data rather than complete sequence data. Ranking using pVAAST p-values instead of LOD scores further improves the accuracy of disease-gene identification, and the improvement is pronounced when the penetrance is low or the phenotypes are ambiguous for a large fraction of the pedigree.

Classic linkage methods have been designed for sparse genetic marker data and model the recombination frequencies between genetic markers and disease to identify large genomic regions in the family that may harbor a causal mutation. In contrast, pVAAST is designed for sequence-based studies and assumes that the causal mutations can be directly assayed. The model can also incorporate an additional unobserved risk locus (latent locus) to capture an additional layer of genetic architecture of the disease, enabling pVAAST to accurately model complex diseases in families with phenocopies or locus heterogeneity. For these reasons, the pVAAST LOD score can outperform the classic two-point parametric LOD score in the scenarios that are evaluated, particularly in challenging scenarios relevant to common, complex disease involving reduced penetrance, locus heterogeneity, small sample size, or ambiguous phenotypes.

An important practical consideration is which family members to sequence in order to achieve optimal power. For rare Mendelian diseases with high penetrance, the choice is straightforward given that the inheritance path of the causal mutation can be inferred. However, for common genetic disorders, the optimal choice of family members is more complex. Sequencing more distantly related individuals increases the number of informative meioses in the pedigree but also increases the probability of phenocopies. Here it is shown that in a common complex disease with a modest level of locus heterogeneity (PAR=0.05 and only 40% of affected individuals carry mutations with odds ratio >1.1 in the gene of interest; sequencing affected 1^{st} or 2^{nd} cousin pairs yields significantly better results than affected parent-offspring pairs in the same family (see FIG. 3). Sequencing the entire extended family offers a modest improvement over cousin pairs, consistent with the findings of¹⁴.

If sample size is not a limiting factor, another consideration is the cost effectiveness of sequencing pedigrees versus unrelated cases. For example, as shown in the dominant simulations in FIG. 2A, pVAAST requires half the number of pedigrees as VAAST but requires two individuals per pedigree to be sequenced. Thus, with affected cousin pairs, the two approaches are equally cost-effective. However, in rare Mendelian diseases with high penetrance, because the p-value increases exponentially with the number of informative meiosis, sequencing affected pairs more distant than the 1^{st} cousin is more cost-effective than sequencing only unrelated index cases from each pedigree. A two-stage design can also be cost effective. Specifically, in the first stage, only unrelated cases are sequenced, and VAAST prioritizes genes according to their significance levels. In the second stage, candidate risk variant in the relatives of affected carriers are genotyped, and pVAAST analyzes the original sequence data with the additional genotype information. This approach can be economical given the relative costs genotyping and whole-exome sequencing.

Existing family-based sequence analysis approaches are typically applicable to only a narrow range of studies. Hard filtering approaches that enforce strict inheritance patterns are appropriate for studies involving small families with rare Mendelian diseases, but do not provide robust statistical interpretations and do not scale to large families or common, complex diseases^{12, 15}. Sequence analysis in large families typically involve multi-step ad hoc procedures in which linkage analysis or IBD mapping is used to identify large genomic regions followed by the application of a series of hard filters based on inheritance patterns, variant annotations, and population allele frequencies¹⁶. In addition, approaches that rely primarily on hard filters do not scale well to multi-family studies^{6, 7}. ASKAT is a family-based rare variant association test that is designed for large multi-family studies, but is not presently applicable to studies involving 20 or fewer small families. Methods used to identify disease-causing de novo mutations can efficiently combine statistical evidence from multiple families, but require parent-offspring trios and cannot incorporate evidence from families with inherited disease¹⁷. In contrast to existing methods, pVAAST performs well across a wide range of study designs, from a single small family with a rare, Mendelian disease to hundreds of families with common, complex genetic diseases and arbitrary pedigree structures. pVAAST is a flexible, general-purpose disease-gene identification tool that combines variant classification, rare-variant association testing, and linkage analysis in a unified statistical framework to increase the power and reduce the technical complexity of family-based sequencing studies.

### Example 14. De Novo Inheritance in an Enteropathy Pedigree

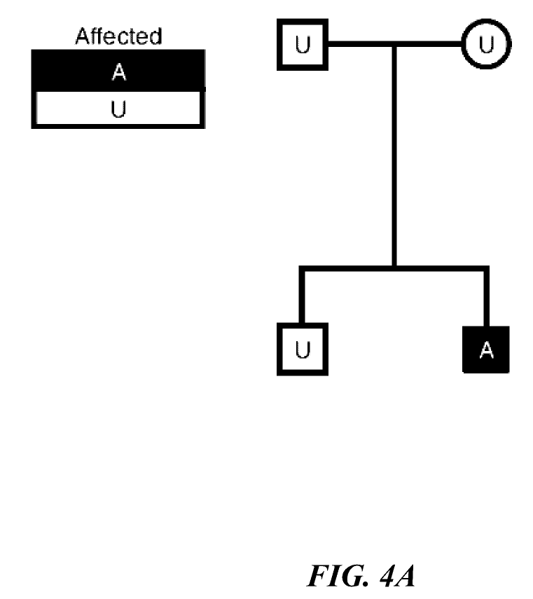

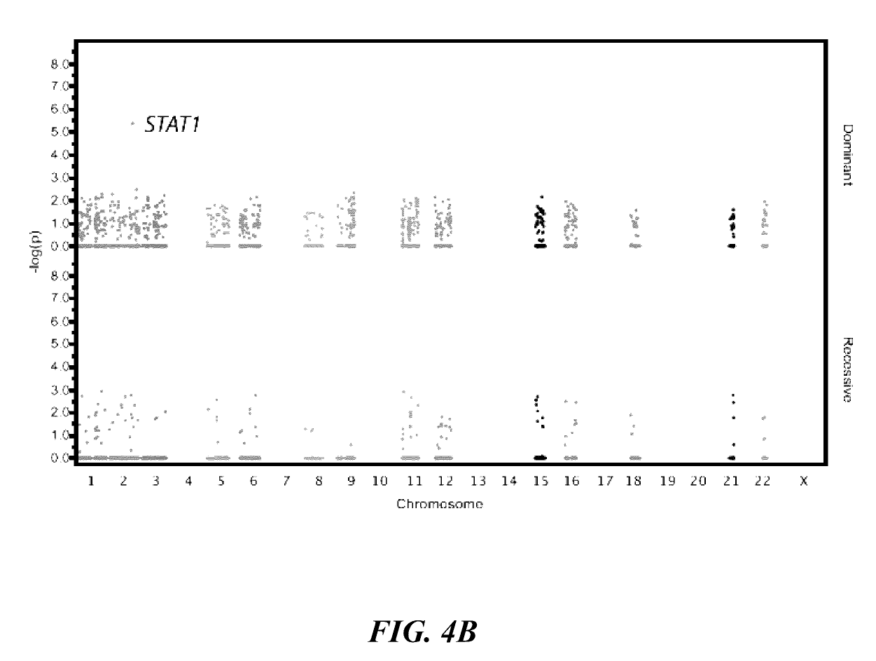

Whole genome sequencing is performed on a family quartet (FIG. 4A), and pVAAST is used to identify the potential causal mutation for a child with undiagnosed enteropathy. The proband is a 12-year-old male with severe diarrhea, total villous atrophy, and hyperthyroidism. Both parents and the sibling of the proband are unaffected. The phenotype is most consistent with the IPEX syndrome, but clinical sequencing of the FOXP3 and IL2RA genes reveal no pathogenic mutations.

The pedigree is analyzed using both the dominant and recessive models in pVAAST. Under the dominant model, the highest-ranking gene is STAT1, which has a P value of 3.97×10⁻⁶. The only variant in this gene is a de novo mutation in the affected child, with a LOD score of 0.70 and a CLRTₚ score of 11.724. The second ranking gene is PAX3 (P=3.33×10⁻³; LOD score=0, and CLRTₚ score=11.047). STAT1 is the only gene in the genome with a LOD score >0.1; genes with LOD score between 0.1 and 0 fit an inheritance pattern of domincance with incomplete penetrance. Under the recessive model, no gene has a P value <1.18×10⁻³ (FIG. 3B). The de novo inheritance pattern is validated by genotyping both the offspring and the parents with Sanger sequencing. Other than this mutation, no exonic variation in STAT1 in the family is observed. This heterozygous mutation is observed only in the proband but not in the parents or unaffected sibling.

The de novo mutation found in the affected child is a single-nucleotide guanine-to-adenine mutation, causing the amino acid change T385M in the DNA-binding motif of STAT1; the reference allele-encoded threonine is conserved among almost all sequenced vertebrate genomes. STAT1 encodes a transcription factor belonging to the signal transducers and activator of transcription family; both gain- and loss-of-function mutations in STAT1 cause human disease. Gain-of-function mutations in STAT1 cause autosomal dominant chronic mucocutaneous candidiasis (CMC) and an IPEX-like phenotype. The T385M mutation has been reported as a cause of CMC in a Japanese patient and a Ukrainian patient. These data support T385M as the causative mutation for this patient's phenotype, and demonstrate pVAAST's ability to identify a causal de novo mutation from a family quartet with a single affected proband.

Methods and systems of the present disclosure can be combined with or modified by other methods and systems, such as those described in Singleton, Marc V., et al. “Phevor Combines Multiple Biomedical Ontologies for Accurate Identification of Disease-Causing Alleles in Single Individuals and Small Nuclear Families,” The American Journal of Human Genetics 94.4 (2014): 599-610 (including Supplemental Data); Hu, Hao, et al. “A unified test of linkage analysis and rare-variant association for analysis of pedigree sequence data.” Nature biotechnology (2014); U.S. Patent Publication Nos. 2007/0042369, 2012/0143512 and 2013/0332081; U.S. Pat. No. 8,417,459; PCT Application No. PCT/US2015/011465; and PCT Publication Nos. WO/2004/092333 and WO/2012/034030, each of which is entirely incorporated herein by reference.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### REFERENCES

1. Jung, J., Weeks, D. E. & Feingold, E. Gene-dropping vs. empirical variance estimation for allele-sharing linkage statistics. Genet Epidemiol 30, 652-665 (2006).
2. Borecki, I. B. & Province, M. A. Linkage and association: basic concepts. Advances in genetics 60, 51-74 (2008).
3. Muller, H. J. Our load of mutations. Am J Hum Genet 2, 111-176 (1950).
4. Wu, M. C. et al. Rare-variant association testing for sequencing data with the sequence kernel association test. Am J Hum Genet 89, 82-93 (2011).
5. Neale, B. M. et al. Testing for an unusual distribution of rare variants. PLoS Genet 7, e1001322 (2011).
6. Yandell, M. et al. A probabilistic disease-gene finder for personal genomes. Genome Res 21, 1529-1542 (2011).
7. Hu, H. et al. VAAST 2.0: Improved Variant Classification and Disease-Gene Identification Using a Conservation-Controlled Amino Acid Substitution Matrix. Genet Epidemiol 37, 622-634 (2013).
8. Schaid, D. J., McDonnell, S. K., Sinnwell, J. P. & Thibodeau, S. N. Multiple genetic variant association testing by collapsing and kernel methods with pedigree or population structured data. Genet Epidemiol 37, 409-418 (2013).
9. Oualkacha, K. et al. Adjusted sequence kernel association test for rare variants controlling for cryptic and family relatedness. Genet Epidemiol 37, 366-376 (2013).
10. Rosner, B. Fundamentals of biostatistics, Edn. 7th. (Brooks/Cole, Cengage Learning, Boston; 2011).
11. Garg, V. et al. GATA4 mutations cause human congenital heart defects and reveal an interaction with TBXS. Nature 424, 443-447 (2003).
12. Roach, J. C. et al. Analysis of genetic inheritance in a family quartet by whole-genome sequencing. Science 328, 636-639 (2010).
13. Metropolis, N., Rosenbluth, A. W., Rosenbluth, M. N., Teller, A. H. & Teller, E. Equation of State Calculations by Fast Computing Machines. J Chem Phys 21, 1087-1092 (1953).
14. Feng, B. J., Tavtigian, S. V., Southey, M. C. & Goldgar, D. E. Design considerations for massively parallel sequencing studies of complex human disease. PloS one 6, e23221 (2011).
15. Wang, K., Li, M. & Hakonarson, H. ANNOVAR: functional annotation of genetic variants from high-throughput sequencing data. Nucleic Acids Res 38, e164 (2010).
16. Marchani, E. E. et al. Identification of rare variants from exome sequence in a large pedigree with autism. Human heredity 74, 153-164 (2012).
17. Heinzen, E. L. et al. De novo mutations in ATP1A3 cause alternating hemiplegia of childhood. Nat Genet 44, 1030-1034 (2012).
18. Elston, R. C. & Stewart, J. A general model for the genetic analysis of pedigree data. Human heredity 21, 523-542 (1971).
19. Fishelson, M. & Geiger, D. Exact genetic linkage computations for general pedigrees. Bioinformatics 18 Suppl 1, S189-198 (2002).
20. Madsen, B. E. & Browning, S. R. A groupwise association test for rare mutations using a weighted sum statistic. PLoS Genet 5, e1000384 (2009).
21. Abecasis, G. R. et al. An integrated map of genetic variation from 1,092 human genomes. Nature 491, 56-65 (2012).
22. Drmanac, R. et al. Human genome sequencing using unchained base reads on self-assembling DNA nanoarrays. Science 327, 78-81 (2010).
23. Li, Y. et al. Resequencing of 200 human exomes identifies an excess of low-frequency non-synonymous coding variants. Nat Genet 42, 969-972 (2010).
24. Reese, M. G. et al. A standard variation file format for human genome sequences. Genome Biol 11, R88 (2010).
25. Database of Single Nucleotide Polymorphisms (dbSNP). Bethesda (Md.): National Center for Biotechnology Information, National Library of Medicine. (dbSNP Build ID: 137). Available from: http://www.ncbi.nlm.nih.goc/SNP/.
26. Exome Variant Server, NHLBI GO Exome Sequencing Project (ESP), Seattle, Wash. (URL: http://evs.gs.washington.edu/EVS/) [March 2013].
27. Wright, S. Evolution in Mendelian populations. 1931. Bull Math Biol 52, 241-295; discussion 201-247 (1990).
28. Dees, N. D. et al. MuSiC: identifying mutational significance in cancer genomes. Genome Res 22, 1589-1598 (2012).
29. Risch, H. A. et al. Prevalence and penetrance of germline BRCA1 and BRCA2 mutations in a population series of 649 women with ovarian cancer. Am J Hum Genet 68, 700-710 (2001).
30. Youlden, D. R. et al. The descriptive epidemiology of female breast cancer: an international comparison of screening, incidence, survival and mortality. Cancer epidemiology 36, 237-248 (2012).

1. A computer system for identifying phenotype-causing genome sequence variants, comprising:
computer memory storing a plurality of genome sequence variants from an assay performed on a biological sample of a subject exhibiting a phenotype, which subject is from a pedigree that comprises said subject and one or more individuals related thereto that do not exhibit said phenotype; and
a computer processor coupled to said computer memory, wherein said computer processor is programmed to:
group said genome sequence variants from said computer memory into user-defined features;
evaluate a potential severity of said genome sequence variants by estimating variant frequency and/or amino acid substitution frequency;
calculate by a summing procedure a likelihood ratio of said genome sequence variants occurring within said user-defined features for said subject as compared to said genome sequence variants occurring within said user defined features in biological samples of control subject(s) not exhibiting said phenotype;
determine log odds (LOD) scores for said genome sequence variants, wherein a given LOD score is indicative of a given genome sequence variant being causative or associated with said phenotype;
prioritize said genome sequence variants by at least said LOD scores, thereby providing prioritized genome sequence variants; and
report said prioritized genome sequence variants.

2. The system of claim 1, wherein said user-defined features comprise one or more of a gene, an exon, an intron, a protein coding sequence, a splice site, a promoter, a regulatory sequence, a protein binding site, an enhancer, and a repressor.

3. The system of claim 1, wherein said genome sequence variants comprise coding and non-coding genome sequence variants, and wherein said computer processor is programmed to (i) score both of said coding and non-coding genome sequence variants; and (ii) evaluate a cumulative impact of both types of said genome sequence variants simultaneously.

4. The system of claim 1, wherein said programmed computer processor is programmed to incorporate both rare and common genomic sequence variants to identify variants that are associated with common phenotypes.

5. The system of claim 1, wherein said phenotype is a disease.

6. The system of claim 1, further comprising a communication interface for obtaining genetic information containing said genome sequence variants of said subject, wherein said computer processor is programmed to use said genome sequence variants to analyze said genetic information of said subject to identify another phenotype in said subject.

7. (canceled)

8. The system of claim 6, wherein said computer processor is programmed to (i) generate a report that is indicative of said another phenotype in said subject, or (ii) wherein said computer processor is programmed to use said prioritized genome sequence variants to identify a disease associated with said phenotype or said another phenotype in said subject.

9. (canceled)

10. (canceled)

11. The system of claim 8, wherein said computer processor is programmed to recommend a therapeutic intervention for said disease.

12. The system of claim 8, wherein said report is provided for display on a user interface on an electronic display.

13. The system of claim 12, wherein said computer processor is programmed to format said report for display on said user interface.

14. A method for identifying phenotype-causing genome sequence variants, comprising:
(a) using a programmed computer processor to (i) group genome sequence variants within user-defined features, which genome sequence variants are from an assay performed on a biological sample of a subject exhibiting a phenotype, which subject is from a pedigree that comprises said subject and one or more individuals related thereto that do not exhibit said phenotype, and (ii) evaluate a potential severity of said genome sequence variants by estimating variant frequency and/or amino acid substitution frequency;
(b) calculating by a summing procedure a likelihood ratio of said genome sequence variants occurring within said user-defined features for said subject as compared to said genome sequence variants occurring within said user defined features in biological samples of control subject(s) not exhibiting said phenotype;
(c) determining log odds (LOD) scores for said genome sequence variants, wherein a given LOD score is indicative of a given genome sequence variant being causative or associated with said phenotype;
(d) prioritizing said genome sequence variants by at least said LOD scores; and
(e) reporting said genome sequence variants prioritized in (d).

15.-18. (canceled)

19. The method of claim 14, further comprising using said genome sequence variants to identify another phenotype in said subject.

20. The method of claim 19, further comprising (i) generating a report that is indicative of said another phenotype in said subject, or (ii) using said prioritized genome sequence variants to identify a disease associated with said phenotype in said subject.

21. (canceled)

22. (canceled)

23. The method of claim 14, further comprising recommending a therapeutic intervention for said disease.

24. The method of claim 14, further comprising incorporating a genetic profile of a single individual, wherein said genetic profile comprises single-nucleotide polymorphisms, a set of one or more genes, an exome or a genome; a genomic profile of one or more individuals analyzed together; or genomic profiles from individuals from a family.

25. The method of claim 14, wherein said prioritizing said genome sequence variants by at least said LOD scores has a statistical power at least 10 times greater than prioritizing said genome sequence variants without said LOD scores.

26.-28. (canceled)

29. The method of claim 14, wherein said determining of said LOD scores for said genome sequence variants utilizes phasing information of said genome sequence variants.

30. The method of claim 14, wherein said subject exhibiting said phenotype and said individuals not exhibiting said phenotype are included in a target and background database, respectively, wherein said target and background databases comprise: (i) genome sequence variants of said subject exhibiting said phenotype and said individuals not exhibiting said phenotype, and (ii) information on family members of said subject, wherein said information comprises whether said family members have exhibited said phenotype.

31. (canceled)

32. (canceled)

33. The method of claim 14, wherein (i) said genome sequence variants are prioritized using a trained algorithm or (ii) said prioritized genome sequence variants are used to generate said trained algorithm.

34. (canceled)

35. (canceled)

36. The method of claim 14, wherein determining said LOD scores further comprises calculating a likelihood of a null model and an alternative model, wherein said models assume independence between nucleotide sites.

37. The method of claim 36, wherein a significance of said likelihood is determined by permuting to control for linkage disequilibrium.

38.-54. (canceled)

55. The method of claim 14, wherein said LOD scores are determined assuming a genome sequence variant that causes said subject to exhibit said phenotype is inherited under a recessive inheritance model, a recessive with complete penetrance inheritance model, a monogenic recessive inheritance model, or a combination thereof.

56.-59. (canceled)

60. The method of claim 14, further comprising constraining an estimated recombination rate to 0 in both null and alternative models for determining said LOD scores.

61. The method of claim 14, wherein a second latent locus is included in said determining said LOD scores using:
Pₙᵤₗₗ(g_{c},gₗ,p|ρ_{c},ρₗ,f_{c},fₗ)=P(g_{c}|f_{c})P(gₗ,p|ρₗ,fₗ).

62. The method of claim 14, wherein determining said LOD scores further comprises utilizing a rate of de novo mutation per meiosis in human genomes.

63. The method of claim 14, further comprising incorporating a disease model that allows for recessive and compound heterozygote patterns of inheritance by estimating a Boolean risk vector of disease causality at each genome sequence variant using a computational optimization technique.

64. (canceled)

65. The method of claim 63, wherein further comprising optimizing
L=ρᵣⁿ^{ₐ}(1−ρᵣ)ⁿ^{_{b}}ρₙⁿ^{_{c}}(1−ρₙ)ⁿ^{_{d}},
wherein “L” is a joint likelihood that said user-defined features contain two or more genome sequence variants that are associated with said phenotype, “ρᵣ” is a probability that an individual with a genotype associated with said phenotype exhibits said phenotype, “ρₙ” is a probability that an individual with a genotype not associated with said phenotype exhibits said phenotype; “nₐ” and “n_{b}” are total numbers of individuals exhibiting said phenotype and individuals not exhibiting said phenotype, each of which have a genotype associated with said phenotype, respectively; “n_{c}” and “n_{d}” are total numbers of individuals with a genotype not associated with said phenotype that exhibit said phenotype and individuals with a genotype not associated with said phenotype that do not exhibit said phenotype, respectively.

66. The method of claim 14, wherein said LOD scores for each of said genome sequence variants are first determined across each of two or more pedigrees before determining which of said genome sequence variants are used to determine said LOD scores in each of said pedigrees.

67. The method in claim 14, further comprising determining a statistical significance of said likelihood ratio and said LOD scores using a combined permutation test and a gene drop simulation.

68. The method in claim 67, wherein said permutation test estimates said statistical significance in said pedigree, wherein a founder in said pedigree may or may not have genome sequence variant data, by repeated sampling of a combined database of genome sequence variants from target and background genomes and randomly assigning said genome sequence variants from target and background genomes to said founder.

69. (canceled)

70. The method in claim 67, wherein said gene drop simulation randomly determines said genome sequence variants in non-founder members of said pedigree using Mendelian rules of inheritance.

71. (canceled)

72. (canceled)

73. The method in claim 14, wherein identity-by-descent information from genome sequence data of individuals within said pedigree is evaluated during said identifying phenotype-causing genome sequence variants.
